(19) United States
(12) Patent Application Publication
(10) Pub. No.: US 2025/0134464 A1
(43) Pub. Date: May 1, 2025

(54) PRESSURE COMPENSATION FOR WRIST-BASED PULSE SPECTROMETRY

(71) Applicant: Garmin International, Inc., Olathe, KS (US)

(72) Inventors: Paul R. MacDonald, Cochrane (CA); Christopher J. Kulach, Cochrane (CA); Kieran W. Armstrong, Cochrane (CA); Tim A. Verschaeve, Cochrane (CA)

(21) Appl. No.: 18/918,231

(22) Filed: Oct. 17, 2024

Related U.S. Application Data
(60) Provisional application No. 63/593,105, filed on Oct. 25, 2023.

(51) Int. Cl. A61B 5/00; A61B 5/1455

(52) U.S. Cl. CPC A61B 5/6843; A61B 5/7235; A61B 5/14551; A61B 2560/0462

(57) ABSTRACT

An electronic fitness device comprises a housing, a first optical transmitter, a second optical transmitter, an optical receiver, and a processor. The housing is positioned partly in contact with a user's skin. The first optical transmitter emits a first optical signal. The second optical transmitter emits a second optical signal. The optical receiver receives the optical signals and generates a first photoplethysmogram (PPG) signal resulting from the received first optical signal and a second PPG signal resulting from the received second optical signal. The processor receives the PPG signals, determines a pressure metric value based on a characteristic of a waveform of the first PPG signal and a characteristic of a waveform of the second PPG signal, and determines a pressure compensation factor based on the determined pressure metric value, the first wavelength corresponding to the first optical signal and the second wavelength corresponding to the second optical signal.

### RELATED APPLICATIONS

The current patent application is a non-provisional utility patent application which claims priority benefit, with regard to all common subject matter, under 35 U.S.C. § 119 (e) of earlier-filed U.S. Provisional Application Ser. No. 63/593,105, filed Oct. 25, 2023, and entitled “PRESSURE COMPENSATION FOR WRIST-BASED PULSE SPECTROMETRY.” The Provisional application is hereby incorporated by reference, in its entirety, into the current patent application.

### FIELD OF THE TECHNOLOGY

Embodiments of the present technology relate to electronic fitness devices that perform pulse spectrometry and provide compensation for pressure of the device on a user's skin as levels of pressure that cause substantial compression of the blood vessels near the area through which the optical signals pass may be undesirable for determining accurate physiological metrics and information based on the resulting PPG signals.

### BACKGROUND

An electronic fitness device may be configured to perform optical pulse spectrometry for cardiac monitoring of a user of the device. The electronic fitness device is typically worn on the user's wrist and includes a housing that contacts the user's skin and applies a pressure thereto. The electronic fitness device also includes a plurality of optical transmitters and optical receivers that are positioned on a bottom wall of the housing. The plurality of optical transmitters may include optical transmitter arrays forming a plurality of optical transmitters, some of which emit light at a different wavelength than the light emitted by other optical transmitters. It is common for conventional electronic fitness devices to incorporate a plurality of transmitter arrays and a plurality of optical receivers at different locations on the bottom wall of the housing, which enables a plurality of optical signals to pass through different paths, in some cases, using different wavelengths of optical signals. Many optical layouts and topologies are known. The plurality of optical transmitters output optical signals of different wavelengths into the user's skin that are reflected to an optical receiver on the bottom wall of the housing after passing through blood vessels under the surface of the skin. The cardiac monitoring may include physiological metrics and information, such as a user's heart rate and pulse oximetry (blood oxygen saturation), determined based on changes to the optical signals as they pass through the blood vessels and are reflected to an optical receiver on the bottom wall of the housing. Many conventional electronic fitness devices include one or more processors that generate photoplethysmogram (PPG) signals associated with the intensity of reflected light and use the generated PPG signals to determine physiological information about the user, including cardiac metrics, such as a user's heart rate and blood oxygen saturation.

The use of optical transmitters and optical receivers for determining blood-related and cardiac physiological metrics and information for a user requires a strong sensor-skin interface to receive PPG signals having a high signal-to-noise ratio (SNR) and determination accurate physiological metrics and information for the user based on those PPG signals. Accordingly, although some compression of the blood vessels may occur near the area through which the optical signals pass from optical transmitters to one or more optical receivers may occur once pressure caused by the housing is sufficient in order for the optical receivers to receive optical signals with adequate SNR level for use by the processor to determine physiological metrics and information for the user based on the associated PPG signals, higher levels of pressure that cause further compression of the blood vessels near the area through which the optical signals pass may be undesirable for the processor determining physiological metrics and information based on the resulting PPG signals. Depending on how tightly the housing is secured to the user's wrist, it is possible the pressure of the housing on the user's skin compressing the wrist and blood vessels in and around the wrist may be undesirable. Conventional electronic fitness devices do not determine a pressure applied to the user's blood vessels in the area where the housing is secured to the user or account (compensate) for that pressure compressing blood vessels when determining cardiac monitoring for the user based on optical signals that pass through the compressed blood vessels.

### SUMMARY

Embodiments of the present technology provide an electronic fitness device configured to perform optical pulse spectrometry for cardiac monitoring of a user of the device and to compute a pressure compensation factor to account for a pressure level of a housing on the user's skin. The electronic fitness device broadly comprises a housing, a first optical transmitter, a second optical transmitter, an optical receiver, and a processor. The housing includes a bottom wall to be positioned at least partly in contact with a user's skin. The housing asserts a pressure on the user's skin once secured to the user. The first optical transmitter is positioned along the bottom wall and is configured to emit a first optical signal having a first wavelength. The first optical signal is directed into the skin of the user. The second optical transmitter is positioned along the bottom wall and is configured to emit a second optical signal having a second wavelength. The second optical signal is directed into the skin of the user. The optical receiver is positioned along the bottom wall and is configured to receive the first optical signal and the second optical signal, with each optical signal being modulated by the skin of the user, and generate a first photoplethysmogram (PPG) signal resulting from the received first optical signal and a second PPG signal resulting from the received second optical signal. The processor is electrically coupled with the optical receiver and is configured to receive the first PPG signal and the second PPG signal, determine a pressure metric value based on a characteristic of a waveform of the first PPG signal and a characteristic of a waveform of the second PPG signal, and determine a pressure compensation factor based on the determined pressure metric value, the first wavelength corresponding to the first optical signal and the second wavelength corresponding to the second optical signal.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present technology will be apparent from the following detailed description of the embodiments and the accompanying drawing figures.

### BRIEF DESCRIPTION OF DRAWINGS

Embodiments of the present technology are described in detail below with reference to the attached drawing figures, wherein:

FIG. 1 is a top view of an electronic fitness device constructed in accordance with various embodiments of the present technology, the electronic fitness device being worn on a user's wrist;

FIG. 2A is a bottom view of the electronic fitness device, illustrating a first configuration of a plurality of optical transmitter arrays, a plurality of optical transmitters and a plurality of optical receivers which form an optical transmitter and receiver assembly on a bottom wall of a housing of the electronic fitness device;

FIG. 2B is a bottom view of the electronic fitness device, illustrating a second configuration of the optical transmitter and receiver assembly;

FIG. 3A is a schematic block diagram of the first configuration of the optical transmitter and receiver assembly, illustrating individual optical transmitters in the optical transmitter array and signal paths between each optical transmitter and associated optical receiver;

FIG. 3B is a schematic block diagram of the second configuration of the optical transmitter and receiver assembly, illustrating individual optical transmitters in the optical transmitter array and signal paths between each optical transmitter and associated optical receiver;

FIG. 4A is a cross sectional view of the electronic fitness device cut along a vertical plane, illustrating an optical transmitter emitting an optical signal through and/or reflected by the user's skin and received by an optical receiver;

FIG. 4B is a side view of the electronic fitness device, illustrating the optical transmitter and receiver assembly including one optical transmitter emitting an optical signal through and/or reflected by the user's skin and received by one optical receiver;

FIG. 5 is a plot of a signal level vs. time for a photoplethysmogram (PPG) signal waveform;

FIG. 6 is a plot of a signal level vs. time for a cardiac component of the PPG signal;

FIG. 7 is a plot of an absorption coefficient vs. wavelength illustrating whole absorption of the optical signal and including a plurality of wavelength bands;

FIG. 8 is a plot of the absorption coefficient vs. wavelength illustrating whole absorption of the optical signal and including a plurality of individual signal wavelengths;

FIG. 9 is a schematic block diagram of various electronic components of the electronic fitness device;

FIG. 10 is an illustration of a first lookup table for determining a pressure compensation factor;

FIG. 11 is a side view of the electronic fitness device, illustrating one optical transmitter emitting an optical signal through and/or reflected by the user's skin and received by two optical receivers along two different paths at two different distances;

FIG. 12 is an illustration of a second lookup table for determining a pressure compensation factor;

FIG. 13A is a plot depicting an expected relationship between a first PMV associated with optical signals having a first wavelength and a second wavelength and a second PMV associated with optical signals having the second wavelength and a third wavelength;

FIG. 13B is a plot depicting a measured relationship between a first PMV associated with optical signals having a first wavelength and a second wavelength and a second PMV associated with optical signals having the second wavelength and a third wavelength for optical signals output by an optical transmitter array and received by an optical receiver, the optical signals traveling along a common signal path; and

FIG. 13C is a plot depicting a measured relationship between a first PMV associated with optical signals having a first wavelength and a second wavelength and a second PMV associated with optical signals having the second wavelength and a third wavelength for optical signals output by one of a first optical transmitter array or a second optical transmitter array and received by an optical receiver, the optical signals traveling along different signal paths.

The drawing figures do not limit the present technology to the specific embodiments disclosed and described herein. The drawings are not necessarily to scale, emphasis instead being placed upon clearly illustrating the principles of the technology.

### DETAILED DESCRIPTION OF THE TECHNOLOGY

The following detailed description of the technology references the accompanying drawings that illustrate specific embodiments in which the technology can be practiced. The embodiments are intended to describe aspects of the technology in sufficient detail to enable those skilled in the art to practice the technology. Other embodiments can be utilized and changes can be made without departing from the scope of the present technology. The following detailed description is, therefore, not to be taken in a limiting sense. The scope of the present technology is defined only by the appended claims, along with the full scope of equivalents to which such claims are entitled.

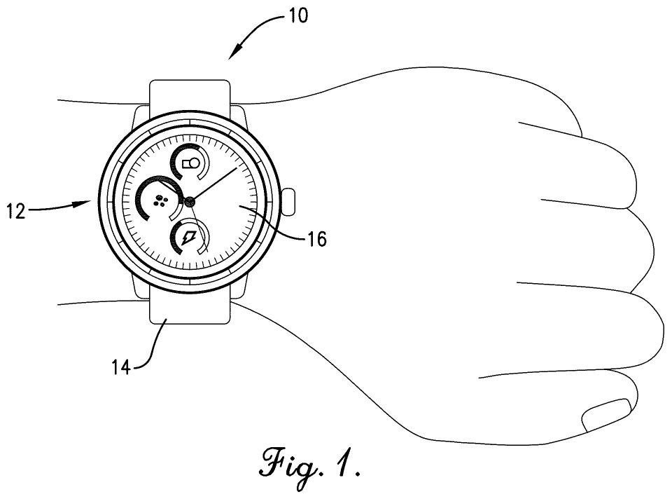

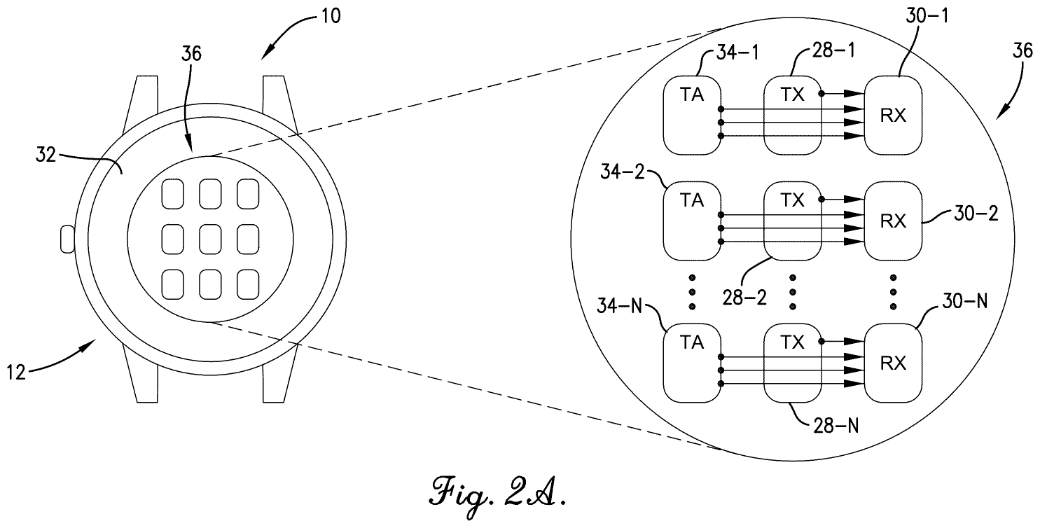

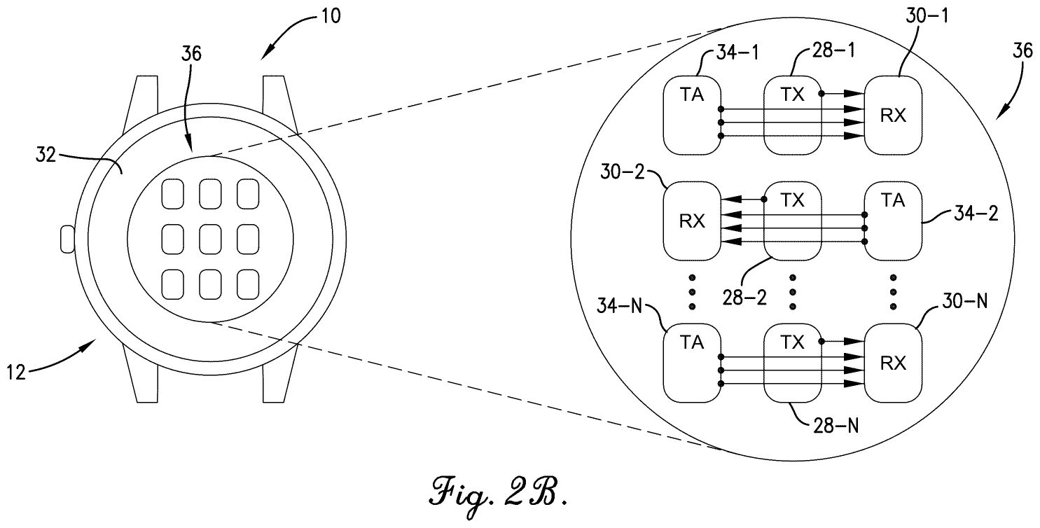

Referring to FIGS. 1, 2A, and 2B, an electronic fitness device is shown. The electronic fitness device performs optical pulse spectrometry for cardiac monitoring of a user of the device. The user (wearer) may be any individual who wears the electronic device such that a housing of the electronic device is located proximate to skin of the individual (e.g., worn against the person's wrist, abdomen, leg, etc.). The cardiac monitoring may include physiological metrics and information such as a user's glycated hemoglobin (HbA1c) level, pulse oximetry level (blood oxygen saturation) or heart rate. The electronic fitness device may utilize a photoplethysmogram (PPG) signal to determine the cardiac monitoring information. The PPG signal is typically output by a photodiode and is commonly utilized to identify changes in the volume of blood in the skin proximate to the photodiode and is collected over a period of time encompassing a plurality of heart beats. The electronic fitness device may include optical devices, such as an optical transmitter, which emits an optical signal (light) into the user's skin, and an optical receiver, which receives reflections of the optical signal (light) from the skin and generates a PPG signal corresponding to the intensity of the received light. Typically, the electronic fitness device includes a housing and straps enabling it to be worn on the user's wrist, arm, leg, or torso, or as a ring worn on a finger of the user and the optical devices are positioned on the back, or bottom wall, of the housing to orient the optical devices to output and receive light from the user's skin when the device is worn.

Referring to FIGS. 2A, 2B, 3A, 3B, 4A, and 4B, many body-worn devices include a housing having a bottom wall retaining optical transmitters (e.g., LEDs) which are labeled “TX” in the figures and that emit (output) light as an optical signal into a user's extremity (e.g., wrist) and optical receivers (photodiodes) which are labeled “RX” in the figures and that receive the optical signal after it has reflected from, and/or transmitted through, blood vessels of the user. The optical signal is modified, influenced or otherwise impacted by reflecting from, and transmitting through, the blood vessels. The optical signals pass through, and/or are reflect from, the user's skin (human tissue), where they are modified, influenced or otherwise impacted, by the amount or flow of blood, or other characteristics of blood vessels, in the signal path of the optical signal. Specifically, the optical signal is modified influenced or otherwise impacted by the blood flow in response to the beating of the user's heart, or the cardiac cycle. After interacting with the user's skin, the optical signals are received by one or more optical receivers of the device. The optical receivers output the analog electronic PPG signal corresponding to the intensity of the optical signal received. The electronic devices use the PPG signals to determine cardiac information about the user, including physiological metrics, such as a user's blood oxygen saturation level or heart rate. A transmitter array may form a plurality of optical transmitters, some of which emit light at a different wavelength than the light emitted by other optical transmitters. It is common for conventional body-worn devices to incorporate a plurality of transmitter arrays and a plurality of optical receivers at different locations, which enables optical signals to pass through many paths and use different wavelengths of light. The techniques used to control the output of optical signals and positioning of optical components may impact the quality of reflections received by the optical sensor(s) and the corresponding PPG signal, which likely impacts the accuracy of measurements and determinations of cardiac information about the user.

An optical transmitter outputs optical signals into skin tissue, which is reflected through scattering back towards one or more optical receivers. As optical signals are modified influenced or otherwise impacted by the blood flow in response to the beating of the user's heart, or the cardiac cycle, optical signals travelling through the skin tissue are variably attenuated due to temporal and other variations in blood density. The temporal variations in blood density result from temporal variations in vascular pressure during a cardiac cycle. Vascular pressure increases during the systolic phase of the cardiac cycle, resulting in an increase of blood density in tissue and decreases during the diastolic phase resulting in a decrease of blood density. Increase in tissue blood density results in increased attenuation of light. Consequently, less light is captured by the receiver during the systolic phase than during the diastolic phase of the cardiac cycle. This temporal variation of captured light is represented by the PPG signal.

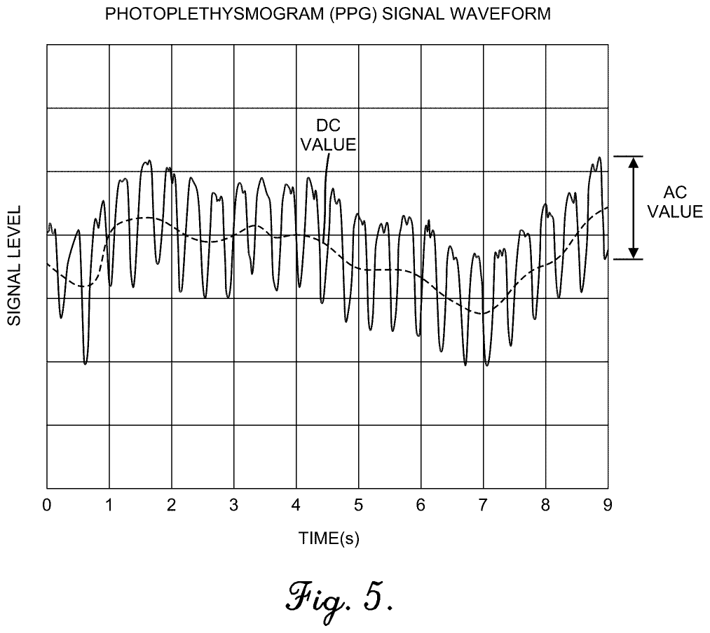

Referring to FIG. 5, a plot of a level of the electrical characteristics, electric voltage and/or electric current, of the PPG signal output from one of the optical receivers is shown. The PPG signal has a periodic waveform, wherein the period corresponds roughly to the user's heart rate, or pulse. The PPG signal also has two components including a direct current (DC) value and an alternating current (AC) value. The DC value may be the instantaneous average value or mean value of the waveform for a moving window of time. Alternatively, the DC value may be the average value or mean value of the local maximum and the local minimum over successive small periods of time of the waveform. The AC value of the waveform is a moving peak-to-peak value, i.e., the local maximum minus the local minimum, over successive small periods of time.

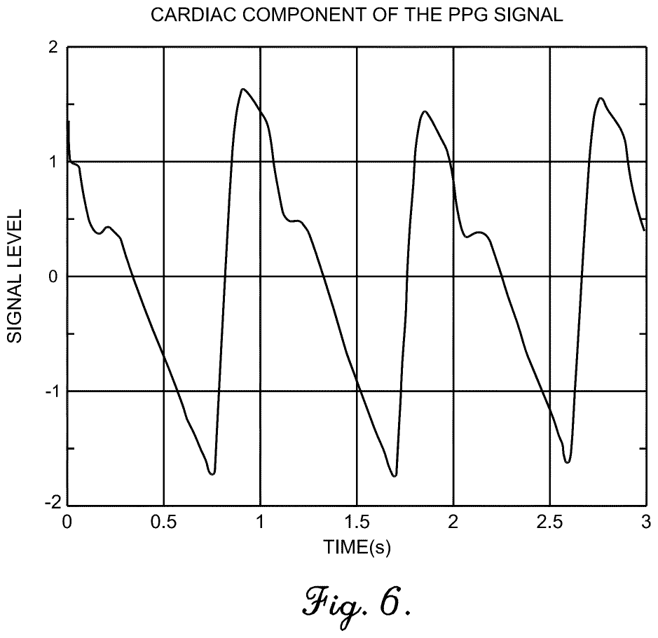

The PPG signal also includes a cardiac component, whose signal level is plotted vs. time in FIG. 6. The cardiac component is typically a periodic, substantially sinusoidal waveform from which metrics related to the user's heart are derived.

Pulse spectroscopy is the observation and analysis of optical signals that are directed at human skin in order to determine blood-related and cardiac physiological metrics and information such as a user's glycated hemoglobin (HbA1c), pulse oximetry (blood oxygen saturation), and heart rate. Red blood cells contain oxygenated hemoglobin (oxyhemoglobin, O2Hb) and deoxygenated hemoglobin (deoxyhemoglobin, HHb). A user's glycated hemoglobin level, also known as HbA1c and A1c, is a volume percentage (vol %) of hemoglobin in an individual's blood that is glycated (i.e., hemoglobin to which glucose is bound or over which glucose is coated). Hemoglobin is a protein in red blood cells that transports oxygen from the lungs to all parts of the body. Once glucose (sugar) is bound or attached to hemoglobin (resulting in glycated hemoglobin), the glucose stays attached to the hemoglobin for the life of that red blood cell, which is typically four months. Accordingly, an individual's glycated hemoglobin level provides a long-term indication of the average blood glucose level for the individual (over the past three months) and is typically not impacted substantially by a single meal or activity. As the level of glycated hemoglobin changes slowly over time, an elevated level of glycated hemoglobin in a person's blood may be an indication of prediabetes or diabetes. In contrast, a user's pulse oximetry level, also known as “pulse ox” or SpO2, is a level of blood oxygen saturation, where a user's pulse oximetry level increases as the concentration of oxygenated hemoglobin increases. Unlike a user's glycated hemoglobin, his or her pulse oximetry levels can change rapidly during activities (almost instantaneously compared to the rate at which glycated hemoglobin levels change).

A healthy (or normal) level of glycated hemoglobin is typically below 5% and levels above 7% or 10%, depending on each individual's health considerations and circumstances, present a concern for developing diabetes, which can also affect the kidneys and other organs of those individuals over the long-term. For someone who has prediabetes or diabetes, determining and monitoring glycated hemoglobin levels can help with the management of glucose and insulin (as it is common for diabetics to maintain a glycated hemoglobin level under 7%). Lowering (and maintaining) the glycated hemoglobin level below 5% is believed to improve and maintain an individual's overall health as elevated levels may be associated with poor control of blood sugar and higher risk of diabetes.

Conventional techniques for determining an individual's glycated hemoglobin level typically involve use of specialized equipment or devices to perform measurements on blood that has been collected from the individual either using a finger stick or collected in a vial (commonly referred to as an “A1c test”). For example, a test may be performed at home or in a laboratory on blood that has been collected using the finger stick, a needle or other process. Accordingly, it would be of great benefit to users of a wrist-worn device having optical transmitters that emit light having certain wavelengths into a user's extremity and optical receivers that receive reflections of the emitted light from the extremity to determine a glycated hemoglobin level or receive general feedback on the overall health of the user on the determined glycated hemoglobin level.

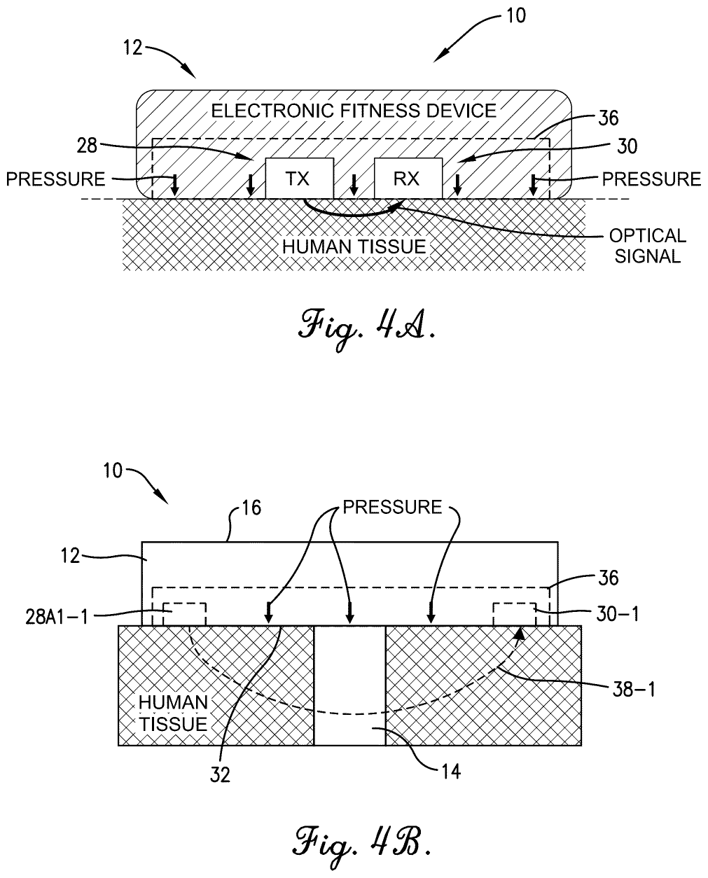

Conventional electronic fitness devices having optical transmitters and optical receivers lack the ability to determine, or account (compensate) for or provide feedback to users regarding a current pressure being applied to the user's skin by the housing. As shown in FIGS. 2A, 2B, 4A, and 4B, a housing including optical transmitters and optical receivers positioned on its bottom wall exerts an amount of force to the skin tissue (indicated as downward-pointing arrows in FIGS. 4A and 4B) that may be the result of numerous factors, including mechanical properties of a strap (band) and a tightness strap (band). Furthermore, a change in extravascular tissue pressure induced by force on a user's skin tissue may be dependent on user-specific body features, such as the location, thickness and size of ligaments and bones in the vicinity of the optical transmitters and optical receivers. Thus, the force associated with the housing of conventional electronic devices induces an unknown amount of extravascular tissue pressure in tissue proximate to the optical transmitters and optical receivers. Consequently, optical receivers of conventional electronic fitness devices, as well as a processor that determines blood-related and cardiac physiological metrics and information for a user, may receive optical signals exhibiting extravascular pressure applied to skin tissue proximate to the optical transmitters and optical receivers that is not determined or accounted for to determine pressure-compensated blood-related and cardiac physiological metrics and information for the user.

Embodiments of the present technology enable a processor to account (compensate) for the application of force to the skin tissue by the optical transmitters and optical receivers when the housing is secured to an extremity of the user. The processor may be configured to account (compensate) for an application of force to the skin tissue by determining a value corresponding to extravascular tissue pressure, such as a multi-wavelength ratio pressure metric, and using the determined multi-wavelength ratio pressure metric to adjust blood-related and cardiac physiological metrics and information determined for a user. In other embodiments, the processor may be configured to account (compensate) for an application of force to the skin tissue by determining a value corresponding to extravascular tissue pressure, such as a path length pulse ratio pressure metric, and using the path length pulse ratio-based pressure metric to adjust blood-related and cardiac physiological metrics and information determined for a user.

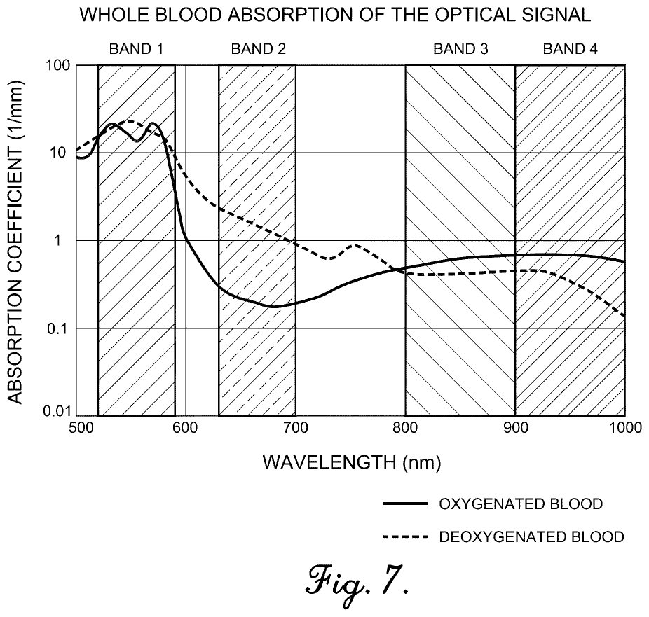

Referring to FIG. 7, a plot of an absorption coefficient versus optical signal wavelength is shown to illustrate whole blood absorption of the optical signal. The plot illustrates the variability of the absorption according to wavelength as well as the differences in absorption between oxygenated blood and deoxygenated blood. Typically, the wavelengths at which the absorption coefficient for oxygenated blood absorption of the optical signal is maximized result in a PPG signal with the cardiac component having an improved signal-to-noise ratio, leading to accurate heart rate determination, than a PPG signal generated from optical signals having other wavelengths. The plot shows a first curve for oxygenated blood and a second curve for deoxygenated blood. The plot further shows a plurality of bands of wavelengths, including a first band (“Band 1”) ranging from approximately 520 nanometers (nm) to approximately 590 nm, a second band (“Band 2”) ranging from approximately 630 nm to approximately 700 nm, a third band (“Band 3”) ranging from approximately 800 nm to approximately 900 nm, and a fourth band (“Band 4”) ranging from approximately 900 nm to approximately 1000 nm. Although four example wavelength bands are shown, it is to be understood that the boundaries of each band could change and implementations in accordance with those variations would be embodiments hereof. The absorption of blood having red blood cell proteins having oxygenated blood (O2Hb) and deoxygenated blood (HHb) is different for optical signals having different wavelengths. Although optical signals in Band 1 correspond to a high absorption level and is visible to the human eye as the color green, the light does not penetrate into some of the lower layers of a user's skin (as light in Band 1 typically remains in the upper layers of the skin). In contrast, light having a wavelength above 600 nm penetrates deeper (lower) layers of the user's skin tissue. In the example above, the emission (output) by optical transmitters of three or more optical signals having a wavelength in Band 2, Band 3 and Band 4 into a user's extremity may enable a processor to determine a physiological metrics and information, such as a user's heart rate, pulse oximetry (blood oxygen saturation) and/or glycated hemoglobin level for the user based on three corresponding PPG signals generated by one or more optical receivers. The optical signals output by the optical transmitters may be visible to the human eye as the color red or invisible to the human eye in the infrared band (or lower microwave band). In embodiments, one optical transmitter may output a first optical signal having a first wavelength in Band 2, a second optical transmitter may output a second optical signal having a second wavelength that is also in Band 2 and a third optical transmitter may output a third optical signal having a third wavelength in Band 4. In other embodiments, three optical transmitters may output a first optical signal having a first wavelength in Band 2, a second optical signal having a second wavelength in Band 3 and third optical signal having a third wavelength in Band 4.

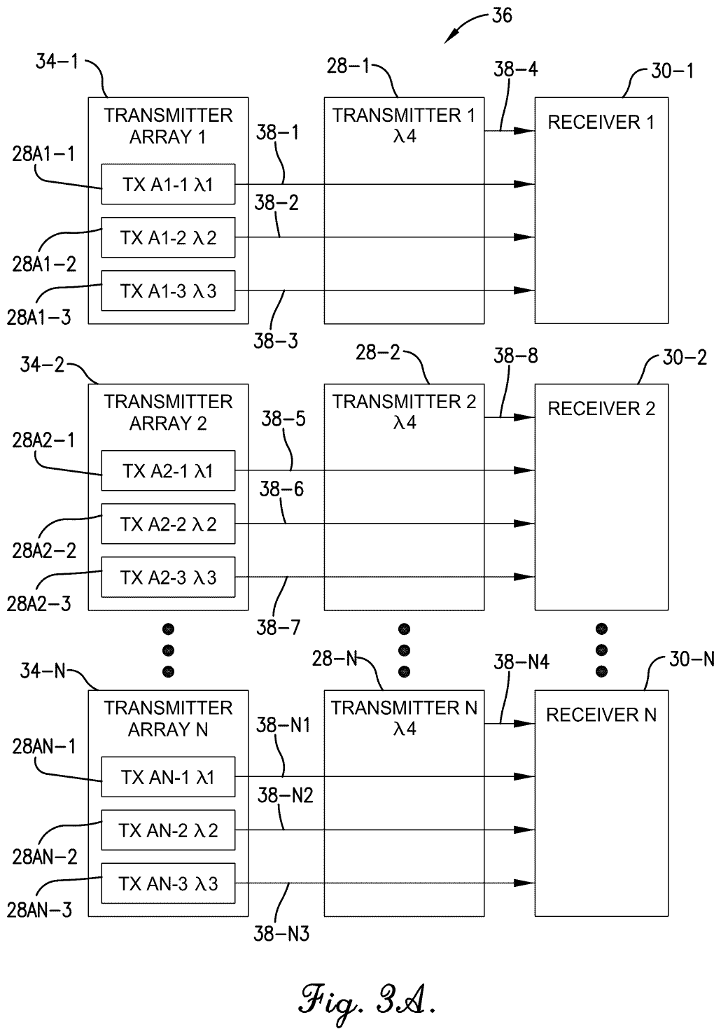

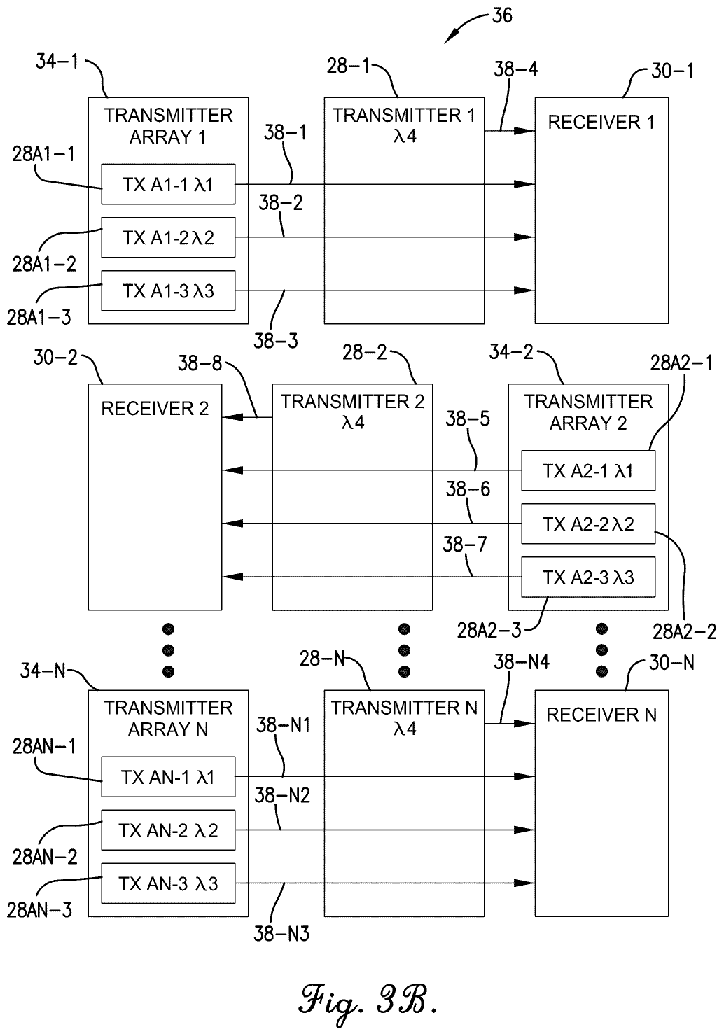

Referring to FIGS. 2A, 2B, 3A, and 3B, various configurations of optical transmitters and optical receivers may transmit and receive optical signals in three or four different wavelength bands. The electronic fitness device may include a plurality of optical transmitter arrays, a plurality of individual optical transmitters, and a plurality of optical receivers. Each optical transmitter array includes a plurality of individual optical transmitters, each transmitting an optical signal having a unique wavelength. For example, each transmitter array may include a first optical transmitter transmitting a first optical signal having a first wavelength (“λ1”), a second optical transmitter transmitting a second optical signal having a second wavelength (“λ2”), and a third optical transmitter transmitting a third optical signal having a third wavelength (“λ3”). In addition, an individual optical transmitter transmitting a fourth optical signal having a fourth wavelength (“λ4”) may be associated, or grouped, with the transmitter array. As discussed above, the four wavelengths may each be in a separate Band identified in FIG. 7 or some wavelengths may share a common Band of FIG. 7.

Exemplary embodiments of the electronic fitness device include a configuration of the optical transmitters and optical receivers as shown in FIGS. 2A and 3A. On the bottom wall of the housing, in each row, one (individual) optical transmitter (depicted in a central column) may be positioned between an optical transmitter array and an optical receiver, resulting in the optical signals output from the center optical transmitter traveling along a shorter signal path to the optical receiver than optical signals output from the optical transmitter array travel to the optical receiver. In each row, one optical receiver is positioned adjacent to the optical transmitter, such that the optical receiver receives one optical signal from the optical transmitter and three optical signals from the optical transmitter array, that is, after each optical signal has traveled through the user's skin and tissue, as shown in FIGS. 4A and 4B. In embodiments, for the purposes of simplicity, the optical transmitter array, the optical transmitter, and the optical receiver in each row operate in combination with one another. However, it is to be understood that any optical receiver proximate to an optical transmitter may receive optical signals output by the optical transmitter. As multiple optical transmitter-receiver combinations may be positioned adjacent to one another (vertically) to form a column, many optical signal paths that are not drawn in FIGS. 2A and 3A may exist. Additionally, it is to be understood that the concept described herein in the context of grouping optical elements by each row may be implemented by grouping optical elements by column. Many known optical layouts and topologies are contemplated and may be utilized.

For example, other embodiments of the electronic fitness device include a configuration of the optical transmitters and optical receivers as shown in FIGS. 2B and 3B, which is a subtle variation of the configuration of FIGS. 2A and 3A. A first group of the optical transmitter-receiver combinations may be positioned in a first orientation, such that the optical receiver is positioned to the right (as shown) of the optical transmitter array and the optical transmitter, and a second group of the optical transmitter-receiver combinations may be positioned in a second orientation, such that the optical receiver is positioned to the left (as shown) of the optical transmitter array and the optical transmitter.

Other configurations of the optical transmitter-receiver combinations are possible than those shown in FIGS. 2A, 2B, 3A, and 3B. For example, the optical transmitter array and the optical transmitter may be spaced farther apart such that the optical receiver may be positioned therebetween. In such a configuration, the optical receiver may receive the three optical signals from the optical transmitter array from a first direction and may receive the one optical signal from the optical transmitter from a second, opposing direction. Furthermore, the optical transmitter-receiver combinations may include a plurality of optical transmitters and a plurality of optical receivers in multiple combinations or permutations.

In embodiments, a processor of the electronic fitness device may determine blood content of one or more dyshemoglobin: carboxyhemoglobin (COHb), methemoglobin (MHb), sulfhemoglobin (SHb) in addition to determining glycated hemoglobin (HbA1c), oxyhemoglobin (O2Hb), deoxyhemoglobin (HHb) and hematocrit (SpHb). For example, in embodiments, determining HHb, O2Hb and glycated hemoglobin (HbA1c) content, at least three PPG signals with different optical signal wavelengths λ1, λ2, λ3 are used to determine HHb content (VHHb), O2Hb content (VO2Hb), and glycated hemoglobin (VHbA1c) content. A ratio of AC to DC (ACRλn) is determined for each wavelength n=1, 2, 3 (EQ. 1). Subsequently, a simultaneous set of linear equations (EQ. 2-4) is solved for content of each component, where cn, kAn, kBn, kCn are empirical or calculated constants. Subsequently, determined values of VHHb, VO2Hb and VhbA1c, are used to determine a fractional blood oxygen saturation (SpO2, EQ. 5), functional blood oxygen saturation (SpO2, EQ. 6), and glycated hemoglobin (HbA1c) (EQ. 7).

$${ACR}_{λ n} = {AC}_{λ n} / {DC}_{λ n} + {c}_{n} [ n = 1 , 2 , 3 ] \qquad EQ . 1$$

$${ACR}_{λ 1} = {V}_{HHb} {k}_{A 1} + {V}_{O 2 Hb} {k}_{B 1} + {V}_{HbA 1 c} {k}_{C 1} \qquad EQ . 2$$

$${ACR}_{λ 2} = {V}_{HHb} {k}_{A 2} + {V}_{O 2 Hb} {k}_{B 2} + {V}_{HbA 1 c} {k}_{C 2} \qquad EQ . 3$$

$${ACR}_{λ 3} = {V}_{HHb} {k}_{A 3} + {V}_{O 2 Hb} {k}_{B 3} + {V}_{HbA 1 c} {k}_{C 3} \qquad EQ . 4$$

$$Fractional SpO 2 = {V}_{O 2 Hb} / ( {V}_{HHb} + {V}_{O 2 Hb} + {V}_{HbA 1 c} ) \qquad Eq . 5$$

$$Functional SpO 2 = {V}_{O 2 Hb} / ( {V}_{HHb} + {V}_{O 2 Hb} ) \qquad EQ . 6$$

$$HbA 1 c = {V}_{HbA 1 c} / ( {V}_{HHb} + {V}_{O 2 Hb} + {V}_{HbA 1 c} ) \qquad EQ . 7$$

In an embodiment, a fourth PPG signal with a fourth optical signal wavelength λ4 is used to determine a heart rate for the user and a fifth PPG signal with a fifth optical signal wavelength λ5 is used as a cardiac signal reference.

As the use of optical transmitters and optical receivers for determining blood-related and cardiac physiological metrics and information for a user requires a strong sensor-skin interface to receive PPG signals having a high signal-to-noise ratio (SNR) and determine accurate physiological metrics and information for the user based on those PPG signals, some compression of the blood vessels may occur near the area through which the optical signals pass from optical transmitters to one or more optical receivers. Compression of the blood vessels may occur once pressure caused by the housing is sufficient in order for the optical receivers to receive optical signals with adequate SNR level for use by the processor to determine physiological metrics and information for the user based on the associated PPG signals. However, higher levels of pressure that cause further compression of the blood vessels near the area through which the optical signals pass may be undesirable for determining accurate physiological metrics and information based on the resulting PPG signals. The housing, which includes optical transmitters (e.g., LEDs) that emit (output) light into a user's extremity (e.g., wrist) and optical receivers (photodiodes) that receive reflections of the emitted light surface, being positioned against or adjacent to the skin necessarily exerts a force on the tissue and higher (upper) layers of the user's skin tissue. This force caused by the housing being secured to the user's body results in an increase of pressure on the skin tissue. If securing the housing to the user's body results in a substantial force applied to the blood vessels near the area through which the optical signals pass from optical transmitters to one or more optical receivers, that force may change the amount by which blood density changes within the tissue during the cardiac cycle, in part due to the extravascular tissue pressure acting against the intravascular pressure induced by the heart. A substantial force exerted by the housing of the electronic device on the skin may impact PPG signal parameters, and specifically the AC/DC ratio. In other words, pressure on the skin tissue due to securing the housing of the electronic device on a user's body may impact or alter AC_{λ1}/DC_{λ1} differently than AC_{λ2}/DC_{λ2} and the extent to which the measurements are impacted is related to the force being applied to the skin tissue. In embodiments, the processor of the electronic fitness device may control a display to present information to the user that may enable a user to ensure the housing is applying a force once secured to the user's wrist that is within a predetermined range that results in a signal-to-noise ratio (SNR) for the PPG signals that exceeds a stored SNR threshold without causing undesired compression of the blood vessels that may adversely impact the accuracy of physiological metrics and information determined by the processor for the user based on the PPG signals.

In order for the processor of the electronic fitness device to account (compensate) for compression of a user's blood vessels near the area through which the optical signals pass from optical transmitters to one or more optical receivers resulting from the application of force to the skin tissue by the optical transmitters and optical receivers when the housing is secured to an extremity of the user, the processor is configured to determine a pressure metric value that corresponds to extravascular tissue pressure. Specifically, the processor may use two PPG signals to determine a pressure metric value based on the characteristics of a first optical signal having a first optical signal wavelength (“λ1”) and a second optical signal having a second optical signal wavelength (“λ2”). In embodiments, the processor, which is electronically coupled to the memory element of the electronic fitness device, may be configured to determine a pressure metric value (PMV) equal to a first quotient of the AC value and the DC value of the first optical signal (having a first optical signal wavelength) divided by a second quotient of the AC value and the DC value of the second optical signal (at a second optical signal wavelength) (EQ. 8) and store the determined PMV in the memory element:

$$Pressure Metric value = \frac{{AC}_{λ 1} / {DC}_{λ 1}}{{AC}_{λ 2} / {DC}_{λ 2}} \qquad EQ . 8$$

In embodiments related to the determination and use of the wavelength ratio pressure metric, λ1 and λ2 of a first optical signal and a second optical signal, respectively, may correspond to wavelengths which optical parameters are relatively responsive to changes in extravascular pressure but substantially unresponsive to changes in blood components. For instance, referring to FIG. 7, λ1 may correspond to a first wavelength in Band 3 and λ2 may correspond to a second wavelength in Band 4. Alternatively, in other embodiments, λ1 and λ2 may both correspond to wavelengths in Band 3. In embodiments, λ1 and λ2 may both correspond to wavelengths in Band 4.

In embodiments, use of optical signals having λ1 and λ2 of 820 nm and 980 nm, respectively, may be appropriate if increased spectral separation between λ1 and λ2 is desired. In other embodiments, λ1 and λ2 have spectral separation of roughly 100 nm between the two wavelengths. In such embodiments, first optical signals having a 940 nm wavelength may be utilized to determine a pulse oximetry level for a user, which could result in the use of second optical signals having a 840 nm wavelength. Accordingly, the pressure metric value corresponding to extravascular tissue pressure may be based on the pressure metric value determined using optical signals having λ1 and λ2 in a first range between 820 nm to 900 nm and a second range between 900 nm and 980 nm, respectively.

The processor is configured to account (compensate) for the application of substantial force to the skin tissue by the electronic device housing, which includes optical transmitters and optical receivers on a bottom wall, when the housing is secured to an extremity of the user due to distortion induced by extravascular pressure using a function of the pressure metric (pm) using a pressure metric that may be determined based on the wavelength ratio pressure metric or the path length pulse ratio pressure metric, as discussed below. Specifically, the processor is configured to compensate for the force to the skin tissue to more accurately determine pressure-compensated physiological metrics and information, such as a pulse oximetry level (blood oxygen saturation) or a glycated hemoglobin (HbA1c) level, for a user where the pressure-compensated physiological metrics and information have one or more of a linear, polynomial, exponential, logarithmic or empirical/numerical functions of the determined pressure metric value.

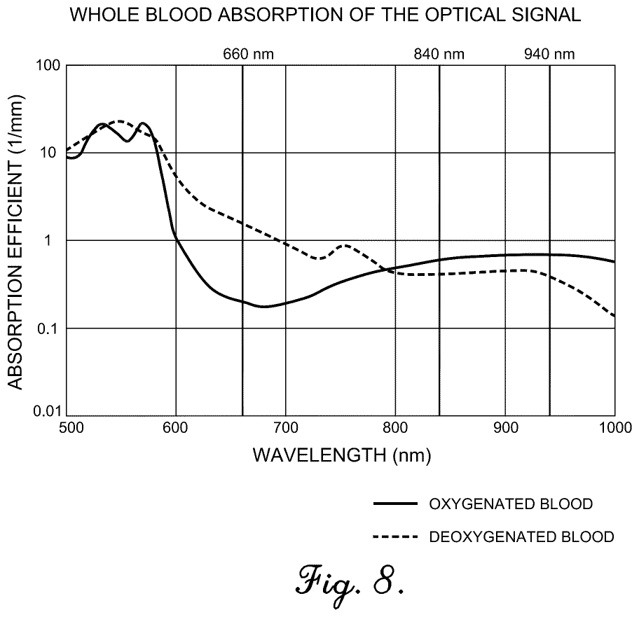

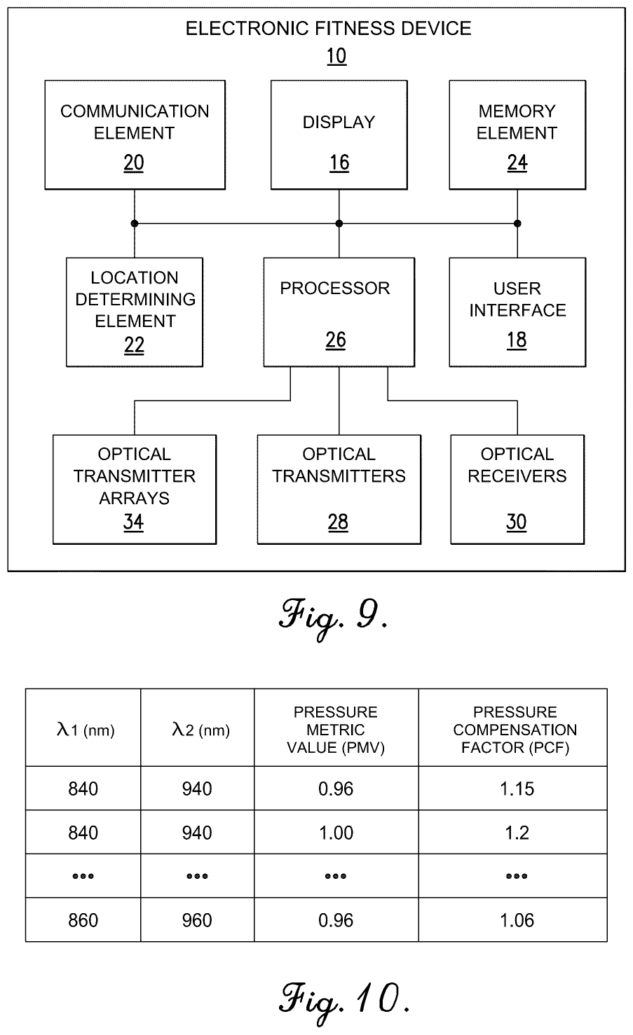

Generally, it is known from determinations of a user's pulse oximetry level (SpO2) that light attenuates at different wavelengths by dominant blood chromophores such as reduced hemoglobin (Hb), oxygenated hemoglobin (HbO2) and water. Known pulse spectrometry techniques, including but not limited to pulse oximetry, utilize optical signals having a wavelength having a maximum difference in attenuation of dominant blood components. Referring to FIG. 8, as the absorption of oxygenated hemoglobin (oxyhemoglobin, O2Hb) and deoxygenated hemoglobin (deoxyhemoglobin, HHb) at 660 nm is appreciably different from the absorption of oxygenated hemoglobin (oxyhemoglobin, O2Hb) and deoxygenated hemoglobin (deoxyhemoglobin, HHb) at 940 nm, both wavelengths are commonly used to determine pulse oximetry for a user. In contrast, attenuation of light in the 820 nm-980 nm range for oxygenated hemoglobin (oxyhemoglobin, O2Hb) and deoxygenated hemoglobin (deoxyhemoglobin, HHb) is substantially constant. Consequently, the relative content of oxygenated hemoglobin (oxyhemoglobin, O2Hb) and deoxygenated hemoglobin (deoxyhemoglobin, HHb) in blood has a substantially equal impact on determinations of pulse-related information for a user based on optical signals having a wavelength within this range. Consequently, metrics based on relative relationships of pulse-related information from two signals in the 820 nm-980 range are substantially insensitive to relative content of oxygenated hemoglobin (oxyhemoglobin, O2Hb) and deoxygenated hemoglobin (deoxyhemoglobin, HHb) in blood. However, use of optical signals having both wavelengths λ1 and λ2 in this range to determine pressure metric value for a user is responsive to the application of extravascular pressure on skin tissue. In embodiments, the processor may be configured to account (compensate) for an application of force to the skin tissue by determining a pressure metric value corresponding to extravascular tissue pressure (EQ. 8) and using the determined pressure metric to adjust blood-related and cardiac physiological metrics and information determined for a user using a pressure compensation factor.

In embodiments, the memory element of the electronic fitness device may store a relationship that associates a pressure compensation factor with a plurality of pressure metric values associated with external skin tissue pressure and a plurality of wavelength sets (λx, λy) of optical signals. For instance, the memory may store a pressure compensation factor for four pressure metric values associated with the pressure applied to a user's extremity (e.g., 3.56 KPa, 5.79 KPa, 8.2 KPa, 10.61 KPa, etc.) and many wavelength sets (λx, λy) between 600 nm and 1000 nm.

$$Pressure Compensation ( λ x , λ x ) = f ( Pressure Metric , λ x , λ y ) \qquad EQ . 9$$

Accordingly, the processor may determine a pressure metric value associated with the pressure applied to a user's extremity and, based on the relationship stored in the memory element, determine a pressure compensation factor for two or more wavelengths of interest, wherein “λx” and “λy” may be any wavelength.

$$Pressure - Compensated Ratio = \frac{{AC}_{λ x} / {DC}_{λ x}}{{AC}_{λ y} / {DC}_{λ y}} + Pressure Compensation ( λ x , λ y ) \qquad EQ . 10$$

For example, the processor may typically utilize the following relationship to determine a pulse oximetry based on another ratio of wavelength-based relationship, where the wavelengths are 660 nm and 940 nm:

$$Pulse Oximetry ( “ SpO 2 ” ) \approx f ( \frac{AC ( 660 ) / DC ( 660 )}{AC ( 940 ) / DC ( 940 )} ) \qquad EQ . 11$$

Once the processor has determined the pressure metric values associated with the pressure applied to a user's extremity (e.g., 3.56 KPa), the processor may determine a pressure compensation factor for the wavelength set (660 nm, 940 nm). The processor may determine a pressure-compensated pulse oximetry based on a pressure compensation factor for 840 nm and/or 940 nm, as shown below:

$$Pressure - Compensated SpO 2 = f ( \frac{AC ( 660 ) / DC ( 660 )}{AC ( 940 ) / DC ( 940 )} + Pressure Compensation ( 840 , 940 ) ) \qquad EQ . 12$$

Accordingly, the processor is configured to account (compensate) for the application of force to the skin tissue by the electronic device housing by determining pressure-compensated physiological metrics and information, such as a pulse oximetry level (blood oxygen saturation) or a glycated hemoglobin (HbA1c) level.

Embodiments of the technology will now be described in more detail with reference to the drawing figures. An exemplary electronic fitness device 10 may be embodied by a smart watch or a fitness band that is typically worn on a user's wrist, but may also be embodied by bands or belts worn on the user's arm, leg, or torso or as a ring around a finger of the user. Other examples of the electronic fitness device 10 may include smartphones, personal data assistants, or the like which include a wall or surface, operable to retain optical devices, that can be pressed against the user's skin. Referring to FIGS. 1, 2A, 2B, 4A, 4B, and 8, the electronic fitness device 10 broadly comprises a housing 12, a wrist band 14, a display 16, a user interface 18, a communication element 20, a location determining element 22, a memory element 24, and a processor 26. The electronic fitness device 10 further comprises a plurality of optical transmitters 28 and a plurality of optical receivers 30 on a bottom wall of the electronic fitness device 10 or any wall of the electronic fitness device 10 that can be pressed against the user's skin.

The housing 12, as shown in FIGS. 1, 2A, 2B, 4A and 4B, generally houses or retains other components of the electronic fitness device 10 and may include or be coupled to the wrist band 14. The housing 12 may include a bottom wall 32, an upper surface, and at least one side wall that bound an internal cavity (not shown in the figures). The bottom wall 32 includes a lower, outer surface that contacts the user's wrist while the user is wearing the electronic fitness device 10. The upper surface opposes the bottom wall 32. In various embodiments, the upper surface may further include an opening that extends from the upper surface to the internal cavity. In some embodiments, such as the exemplary embodiments shown in the figures, the bottom wall 32 of the housing 12 may have a round, circular, or oval shape, with a single circumferential side wall. In other embodiments, the bottom wall 32 may have a four-sided shape, such as a square or rectangle, or other polygonal shape, with the housing 12 including four or more sidewalls. Referring to FIGS. 2A, 2B, 4A and 4B, the bottom wall 32 may include one or more openings in which the plurality of optical transmitters 28 and the plurality of optical receivers 30 are placed, positioned, or located. The one or more openings within the bottom wall 32 may be covered by one or more lenses (not shown in the figures) through which the optical signal may be transmitted and received.

The display 16, as shown in FIG. 1, generally presents the information mentioned above, such as time of day, current location, and the like. The display 16 may be implemented in one of the following technologies: light-emitting diode (LED), organic LED (OLED), Light Emitting Polymer (LEP) or Polymer LED (PLED), liquid crystal display (LCD), thin film transistor (TFT) LCD, LED side-lit or back-lit LCD, or the like, or combinations thereof. In some embodiments, the display 16 may have a round, circular, or oval shape. In other embodiments, the display 16 may possess a square or a rectangular aspect ratio which may be viewed in either a landscape or a portrait orientation.

The user interface 18 generally allows the user to directly interact with the electronic fitness device 10 and may include pushbuttons, rotating knobs, crowns, or the like. In various embodiments, the display 16 may also include a touch screen occupying the entire display 16 or a portion thereof so that the display 16 functions as at least a portion of the user interface 18. The touch screen may allow the user to interact with the electronic fitness device 10 by physically touching, swiping, or gesturing on areas of the display 16.

The communication element 20 generally allows communication with external systems or devices. The communication element 20 may include signal and/or data transmitting and receiving circuits, such as antennas, amplifiers, filters, mixers, oscillators, digital signal processors (DSPs), and the like. The communication element 20 may establish communication wirelessly by utilizing radio frequency (RF) signals and/or data that comply with communication standards such as cellular 2G, 3G, 4G, LTE, or 5G, Institute of Electrical and Electronics Engineers (IEEE) 802.11 standard such as Wi-Fi, IEEE 802.16 standard such as WiMAX, Bluetooth™, or combinations thereof. In addition, the communication element 20 may utilize communication standards such as ANT, ANT+, Bluetooth™ low energy (BLE), the industrial, scientific, and medical (ISM) band at 2.4 gigahertz (GHz), or the like. Alternatively, or in addition, the communication element 20 may establish communication through connectors or couplers that receive metal conductor wires or cables which are compatible with networking technologies such as Ethernet. In certain embodiments, the communication element 20 may also couple with optical fiber cables. The communication element 20 may be in electronic communication with the memory element 24 and the processor 26.

The location determining element 22 generally determines a current geolocation of the electronic fitness device 10 and may receive and process radio frequency (RF) signals from a multi-constellation global navigation satellite system (GNSS) such as the global positioning system (GPS) utilized in the United States, the Galileo system utilized in Europe, the GLONASS system utilized in Russia, or the like. The location determining element 22 may accompany or include an antenna to assist in receiving the satellite signals. The antenna may be a patch antenna, a linear antenna, a loop antenna, a slot antenna, or any other type of antenna that can be used with location or navigation devices. The location determining element 22 may include satellite navigation receivers, processors, controllers, other computing devices, or combinations thereof, and memory. The location determining element 22 may process a location electronic signal communicated from the antenna which receives the location wireless signal from one or more satellites of the GNSS. The location wireless signal includes data from which geographic information such as the current geolocation is derived. The current geolocation may include coordinates, such as the latitude and longitude, of the current location of the electronic fitness device 10. The location determining element 22 may communicate the current geolocation to the processor 26, the memory element 24, or both.

Although embodiments of the location determining element 22 may include a satellite navigation receiver, it will be appreciated that other location-determining technology may be used. For example, cellular towers or any customized transmitting radio frequency towers can be used instead of satellites may be used to determine the location of the electronic fitness device 10 by receiving data from at least three transmitting locations and then performing basic triangulation calculations to determine the relative position of the device with respect to the transmitting locations. With such a configuration, any standard geometric triangulation algorithm can be used to determine the location of the electronic fitness device 10. The location determining element 22 may also include or be coupled with a pedometer, accelerometer, compass, or other dead-reckoning components which allow it to determine the location of the electronic fitness device 10. The location determining element 22 may determine the current geographic location through a communications network, such as by using Assisted GPS (A-GPS), or from another electronic fitness device. The location determining element 22 may even receive location data directly from a user.

The memory element 24 may be embodied by devices or components that store data in general, and digital or binary data in particular, and may include exemplary electronic hardware data storage devices or components such as read-only memory (ROM), programmable ROM, erasable programmable ROM, random-access memory (RAM) such as static RAM (SRAM) or dynamic RAM (DRAM), cache memory, hard disks, optical disks, flash memory, thumb drives, universal serial bus (USB) drives, solid state memory, or the like, or combinations thereof. In some embodiments, the memory element 24 may be embedded in, or packaged in the same package as, the processor 26. The memory element 24 may include, constitute, or embody, a non-transitory “computer-readable medium”. The memory element 24 may store the instructions, code, code statements, code segments, software, firmware, programs, applications, apps, services, daemons, or the like that are executed by the processor 26. The memory element 24 is in electronic communication with the processor 26 and may also store the optical signals output by each optical receiver, a carrier frequency corresponding to each optical transmitter (and optical signal output by the optical transmitter) and data that is received by the processor 26 or the device in which the processor 26 is implemented. The processor 26 may further store data or intermediate results generated during processing, calculations, and/or computations as well as data or final results after processing, calculations, and/or computations. In addition, the memory element 24 may store settings, databases, and the like.

The processor 26 may comprise one or more processors. The processor 26 may include electronic hardware components such as microprocessors (single-core or multi-core), microcontrollers, digital signal processors (DSPs), field-programmable gate arrays (FPGAs), analog and/or digital application-specific integrated circuits (ASICs), intelligence circuitry, or the like, or combinations thereof. The processor 26 may generally execute, process, or run instructions, code, code segments, code statements, software, firmware, programs, applications, apps, processes, services, daemons, or the like. The processor 26 may also include hardware components such as registers, finite-state machines, sequential and combinational logic, configurable logic blocks, and other electronic circuits that can perform the functions necessary for the operation of the present technology. In certain embodiments, the processor 26 may include multiple computational components and functional blocks that are packaged separately but function as a single unit. In some embodiments, the processor 26 may further include multiprocessor architectures, parallel processor architectures, processor clusters, and the like, which provide high performance computing. The processor 26 may be in electronic communication with the other electronic components of the electronic fitness device 10 through serial or parallel links that include universal busses, address busses, data busses, control lines, and the like. In addition, the processor 26 may include analog to digital converters (ADCs) to convert analog electronic signals to sampled digital data values, or streams of sampled digital data values, and/or digital to analog converters (DACs) to convert digital data values, or streams of digital data values, to analog electronic signals.

The processor 26 receives input from the user through the user interface 18 regarding one or more of the physiological metrics the user would like to have determined. In addition, the processor 26 may execute a program or application that automatically determines one or more of the physiological metrics on a periodic basis.

The processor 26 outputs a plurality of transmitter electronic signals, wherein each transmitter electronic signal is communicated to, or received by, a respective one of the optical transmitters 28. The transmitter electronic signal has a first (nonzero) DC level to turn the optical transmitter 28 on and a second DC level (usually approximately 0 Volts) to turn the optical transmitter 28 off. As discussed in more detail below, each optical transmitter 28 emits an optical signal at a specific wavelength. The processor 26 further determines which one or more of the optical transmitters 28 will receive the transmitter electronic signals according to one or more wavelengths associated with the desired physiological metric to determine. For example, if pulse oximetry is desired, then, according to EQ. 11, wavelengths of 660 nm and 940 nm are required. Thus, processor 26 outputs transmitter electronic signals to the optical transmitters 28 configured or operable to emit first optical signals having a wavelength of 660 nm and second optical signals having a wavelength of 940 nm.

The processor 26 receives a plurality of PPG (electronic) signals, with each PPG signal being received from a respective one of the optical receivers 30. Each optical receiver 30 receives reflections of the optical signal from the skin and generates a PPG signal corresponding to the intensity of the received light.

The processor 26 analyzes and processes the data from each PPG signal, wherein the PPG signal is converted to digital data, or a stream of sampled digital data values, by one or more ADCs integrated with the processor 26, integrated with the optical receivers 30, or implemented between the optical receivers 30 and the processor 26. Using the PPG signal data, the processor 26 is configured to determine an AC value and a DC value of the PPG signal waveform, as indicated in FIG. 5. The AC value is determined or calculated as a moving peak-to-peak value or a difference between a local maximum value of the PPG signal data and a local minimum value of the PPG signal data. In some embodiments, the AC value may be calculated every time a new local maximum value or a new local minimum value is determined. The DC value is determined or calculated as an instantaneous average value or mean value of the PPG signal data for a moving window of time. Alternatively, the DC value is determined or calculated as the average value or mean value of the local maximum and the local minimum over successive small periods of time of the PPG signal data. The AC value and the DC value are determined for each of the PPG signals received by the processor 26. That is, the processor 26 is configured to determine the AC value and the DC value for the PPG signals resulting from the optical signals having a wavelength, λ, in one or more of the four wavelength bands shown in FIG. 7.

Once the AC value and the DC value for at least two PPG signals resulting from optical signals having different wavelengths, that is ACλ1, DCλ1, ACλ2, and DCλ2, is determined, the processor 26 is configured to determine or calculate a pressure metric value (PMV) as given in EQ. 8. As an example, the processor 26 may be configured to utilize the PPG signals resulting from first optical signals having a wavelength of 840 nm and second optical signals having a wavelength of 940 nm. Using the data from the two PPG signals, the processor 26 is configured to determine AC840 nm, DC840 nm, AC940 nm, and DC940 nm. The processor 26 uses those values in EQ. 8 to determine PMV840,940.

The two wavelengths, λ1 and λ2, and the determined pressure metric value (PMV) are utilized by the processor to determine a pressure compensation factor (PCF). Referring to FIG. 10, in some embodiments, a virtual lookup table or database is stored in the memory element 24, wherein the lookup table includes four columns and a plurality of rows of data. The four columns include the first wavelength λ1, the second wavelength λ2, the pressure metric value (PMV), and the pressure compensation factor (PCF). Each row includes a PCF value that is associated with a combination of respective values of the first wavelength λ1, the second wavelength λ2, and the PMV. The first wavelength λ1, the second wavelength λ2, and the PMV are used as inputs, keys, or independent variables to substantially match the respective values in a given row of the table. The PCF, associated with the respective values of the first wavelength λ1, the second wavelength λ2, and the PMV, is read out from the memory element 24. While the values of the first wavelength λ1 and the second wavelength λ2 may match respective values in columns 1 and 2 of the lookup table, it is possible that the determined value of the PMV may not match any associated values in the third column. In some instances, the processor 26 may choose the PCF associated with the stored value of the PMV that most closely matches the determined value of the PMV. In other instances, the processor 26 may interpolate the PCF according to the difference between the determined value of the PMV and the closest stored value of the PMV. For example, if the determined value of the PMV is larger or smaller than the stored value of the PMV by a certain ratio or proportion, then the processor 26 may determine an interpolated value of the PCF that is larger or smaller than the stored PCF by the same ratio or proportion.

In other embodiments, processor 26 is configured to apply a mathematical relationship between the PCF and the first wavelength λ1, the second wavelength λ2, and the PMV, the mathematical relationship stored in the memory element 24. In such embodiments, the processor 26 is configured to use the first wavelength λ1, the second wavelength λ2, and the PMV as inputs to one or more mathematical equations and the one or more mathematical equations are solved to determine the PCF. Alternatively, the processor 26 is configured to implement a multi-step algorithm in which the first wavelength λ1, the second wavelength λ2, and the PMV are used or entered to determine the PCF.

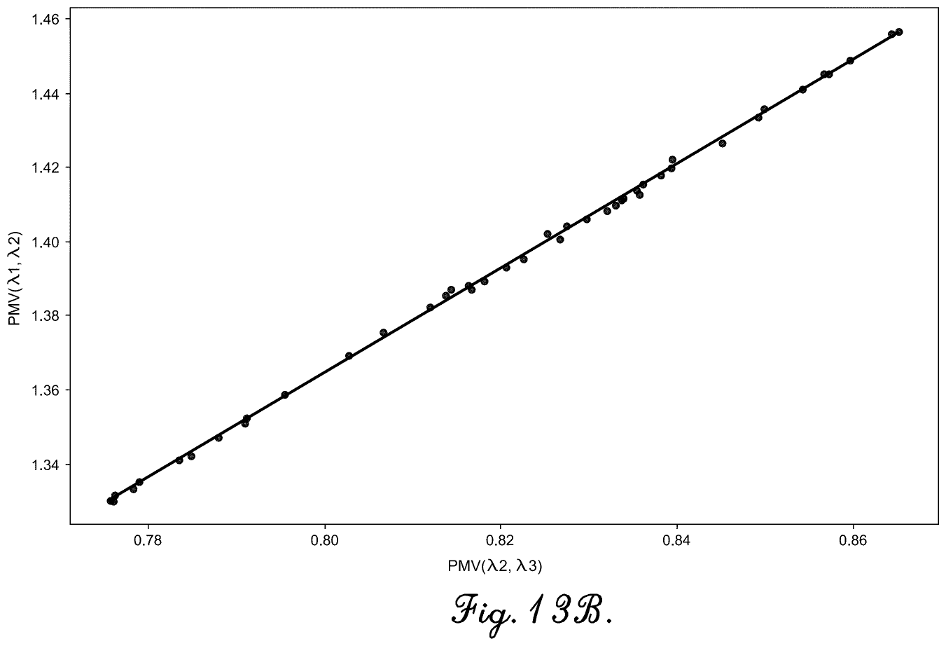

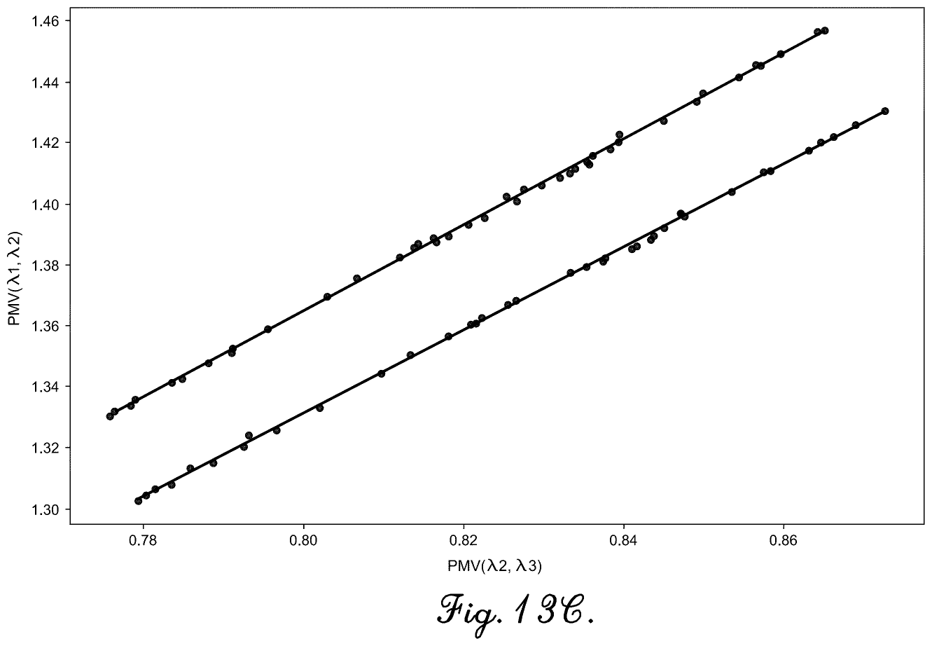

In some embodiments, the processor 26 is configured to determine the PCF based on a stored relationship of the variability of blood oxygenation (SpO2) corresponding to at least two PMVs (EQ. 8) for at least three wavelengths of optical signals (in two wavelength pairs) and a measured relationship of the variability of blood oxygenation (SpO2) corresponding to at least two PMVs (EQ. 8) for the at least three wavelengths of optical signals (for the two wavelength pairs). The stored relationship of PMVs for two wavelength pairs is over a range of variation in blood oxygenation (SpO2) may be based on a typical or expected variability for the user based on physiological information, such as age, weight and other characteristics. As stated above, a user's pulse oximetry level (SpO2) is a level of blood oxygen saturation that changes (increases or decreases) continuously as the concentration of oxygenated hemoglobin changes (increases or decreases) and, unlike a user's glycated hemoglobin, his or her pulse oximetry levels can change rapidly during activities or any other period of time. In fact, for some individuals, the natural range of the continuous changes in pulse oximetry level (SpO2) in blood (within a few seconds or minutes) may be as great as ten percent (10%). Accordingly, changes in SpO2 levels may affect mean blood attenuation of oxygenated hemoglobin (HbO2) and reduced hemoglobin (Hb) at many (and possibly even all) of the wavelengths shown in the plot of the absorption coefficient vs. wavelength illustrating whole absorption of the optical signal of FIG. 8. In embodiments, the memory element 24 may store an expected set of PMVs (EQ. 8) for each wavelength pair associated with optical signals and an expected relationship between the sets of PMVs (e.g., a relationship between a first set of PMVs and a second set of PMVs). For example, the memory element 24 may store an expected first set of PMVs (EQ. 8) over a range of pulse oximetry levels (SpO2) for a first pair of optical signals having the first wavelength λ1 and optical signals having the second wavelength λ2 as well as a second set of PMVs (EQ. 8) over the same range of pulse oximetry levels (SpO2) for a second pair of optical signals having a third wavelength λ3 and optical signals having the first wavelength λ1, which are common to both wavelength pairs. The memory element 24 may also store a relationship between the first set of PMVs associated with the first wavelength λ1 and the second wavelength λ2 and the second set of PMVs associated with the third wavelength λ3 and the second wavelength λ2. In some embodiments, that relationship between the first set of PMVs and the second set of PMVs is categorized by physiological information for each user, such as a user's age, weight and other characteristics. FIG. 13A presents a visualization, over a range of pulse oximetry levels (SpO2) (e.g., between 80% and 99% SpO2), of a stored relationship of a first set of PMVs associated with a first wavelength λ1 and a second wavelength λ2 and a second set of PMVs associated with a third wavelength λ3 and the second wavelength λ2. In embodiments, the processor 26 is configured to receive three PPG signals over a period of time (each PPG signal associated with optical signals having a first wavelength λ1, optical signals having second wavelength λ2 or optical signals having a third wavelength λ3), determine a first set of PMVs associated with PPG signals having the first wavelength λ1 and PPG signals having the second wavelength λ2 and a second set of PMVs associated with PPG signals having the third wavelength λ3 and PPG signals having the second wavelength λ2, determine a measured relationship between the first set of PMVs and the second set of PMVs and then determine a PCF based on the stored relationship and the measured relationship between the first set of PMVs and the second set of PMVs. As a result, in embodiments, the processor 26 is configured to utilize the relationships between PMVs as a current pulse oximetry level (SpO2) changes to determine the PCF.

As an example of using the lookup table with the values from the PMV determination example to determine the PCF, the processor 26 searches the lookup table using values of 840 and 940 to match values in the first and second columns, respectively. The processor 26 also uses the determined value of PMV840,940 to match the value in the third column. The processor 26 then retrieves the value of the PCF from the fourth column of the row that corresponds to the values of the first wavelength λ1, the second wavelength λ2, and the PMV match, or nearly match. The processor 26 may perform an interpolation of the PCF value, if necessary.

Once the processor 26 has determined the PCF, the processor 26 is configured to determine a pressure-compensated physiological metric as a mathematical function of the physiological metric (without compensation) and the PCF. In some embodiments, the mathematical function may be multiplication, such that the pressure-compensated physiological metric is equal to the (uncompensated) physiological metric times (a product of) the PCF. In other embodiments, the mathematical function may be addition, such that the pressure-compensated physiological metric is equal to the (uncompensated) physiological metric plus (added to or subtracted from) the PCF. For example, if the desired physiological metric is pulse oximetry, then processor 26 first determines a value of the pulse oximetry (without any pressure compensation) by outputting transmitter electronic signals to control the optical transmitters 28 configured or operable to emit optical signals having a wavelength of 660 nm and optical signals having a wavelength of 940 nm, which correspond to wavelengths in Band 2 and Band 4 of FIG. 7, as well as optical signals having a wavelength of 840 nm, which corresponds to Band 3 of FIG. 7. The processor 26 receives from an optical receiver 30 the respective PPG signals associated with optical signals having the 660 nm wavelength and optical signals having the 940 nm wavelength and, based on an assessment of those PPG signals, determines the pulse oximetry according to EQ. 11. The processor 26 also receives from the optical receiver 30 the respective PPG signal associated with optical signals having the 840 nm wavelength and, based on an assessment of the PPG signals associated with 840 nm and PPG signals associated with 940 nm, determines the PMV according to EQ. 8. The processor 26 then determines the PCF according to the first wavelength λ1 (840 nm), the second wavelength λ2 (940 nm), and the determined PMV using one of the methods discussed above. For example, the processor 26 may search the lookup table using values of 840 nm and 940 nm to substantially match values in the first and second columns, respectively. The processor 26 also may use the determined value of PMV840,940 to match the value in the third column. The processor 26 may retrieve the corresponding value of the PCF from the fourth column of the row where the values of the first wavelength λ1, the second wavelength λ2, and the PMV match, or substantially (nearly) match. The processor 26 may perform an interpolation of the PCF value, if necessary. With the (uncompensated) pulse oximetry and the PCF each being determined, the processor 26 is configured to determine the pressure-compensated pulse oximetry as a mathematical function of the two values, such as a function of a product of the pulse oximetry and the PCF or a function of a sum of the pulse oximetry and the PCF.

Each optical transmitter 28 (“TX” in the figures) includes an electronically controlled photon generating device such as a light emitting diode (LED), or other short wavelength EM radiation emitting device, that is configured to receive an electronic signal (typically of controlled electric current) and transmit, or output (emit), an optical signal including light, i.e., EM radiation corresponding in amplitude and frequency to the electronic signal and having a wavelength λ in the violet, visible light, or infrared spectrum. The photonic generator of each optical transmitter transmits or outputs electromagnetic radiation having a particular wavelength (the optical signal) in the visible light spectrum, which is typically between approximately 400 nanometers (nm) to 700 nm, in the near infrared spectrum, which is typically between approximately 700 nm to 1,000 nm, or in a wavelength range of 1,000 nm to 1,500 nm. The wavelength of the optical signal is generally determined by, or varies according to, the material from which the photonic generator of each optical transmitter is formed. For instance, optical signals having a wavelength between 500 nm and 600 nm are associated with visually appearing as the color green. In some embodiments, some of the optical transmitters 28 may output optical signals having the same wavelength. In other embodiments, each of the plurality of optical transmitters 28 may output optical signals having a different wavelength. Each optical transmitter 28 may further include a signal amplifier such as a transconductance amplifier configured to drive the LED, wherein the transconductance amplifier converts an input voltage to an output current. Each optical transmitter 28 receives the transmitter electronic signal from the processor 26, which turns the optical transmitter 28 on and turns the optical transmitter 28 off.

Referring to FIGS. 2A, 2B, 3A, and 3B, a plurality of optical transmitters 28 may be packaged together, or positioned in close proximity to one another, to form an optical transmitter array 34. Exemplary embodiments of the optical transmitter array 34 (“TA” in the figures) include a first optical transmitter 28 transmitting a first optical signal having a first wavelength (“λ1”), a second optical transmitter 28 transmitting a second optical signal having a second wavelength (“λ2”), and a third optical transmitter 28 transmitting a third optical signal having a third wavelength (“λ3”).

Each optical receiver 30 includes an electronic device such as a photodiode, a photo resistor, a phototransistor, or the like which is configured to output, or change, an electrical characteristic according to the intensity of the EM radiation, i.e., the optical signal, impinging the device. For example, each optical receiver 30 receives the optical signal from the skin that has been modified by the user's blood vessels and outputs an electric current that corresponds to (varies according to) the intensity of the received optical signal. In addition, the optical receiver 30 is sensitive to the EM radiation from a range of wavelengths including wavelengths in the violet, visible light, and infrared spectrums. Each optical receiver 30 may further include a signal amplifier such as a transimpedance amplifier configured to output the PPG signal, wherein the transimpedance amplifier converts an input current to an output voltage. Furthermore, the PPG signal output by each optical receiver 30 is received by the processor 26.

Referring to FIGS. 2A, 2B, and 4B, a combination of a plurality of optical transmitter arrays 34, a plurality of optical transmitters 28, and a plurality of optical receivers 30 forms an optical transmitter and receiver assembly 36. The optical transmitter and receiver assembly 36 is located on or within a bottom wall 32 of the wrist-worn electronic device 10 such that it is positioned adjacent to skin of the user when the housing 12 is secured to the user's wrist by wrist band 14. The optical transmitter and receiver assembly 36 includes a plurality of optical transmitters 28, a plurality of optical transmitter arrays 34 and a plurality of optical receivers 30. The optical transmitter and receiver assembly 36, optical transmitter arrays 34 and optical receivers 30 are each depicted in FIG. 4B by dashed lines because the optical transmitter and receiver assembly 36, optical transmitter arrays 34 and optical receivers 30 are positioned within and/or at the bottom wall 32 of the wrist-worn electronic device 10.

In FIG. 4B, one of the plurality of optical transmitters arrays 34 and one of the plurality of optical receivers 30 are depicted. For example, a first optical transmitter 28A1-1 (within first optical transmitter array 34-1) is positioned at a first location on the bottom wall 32 and is operable to output a first optical signal that passes through a user's skin. The first optical receiver 30-1 is positioned at a second location on the bottom wall 32 and is operable to receive the first optical signal from the first optical transmitter 28A1-1 such that the optical signals travel along a first signal path 38-1 from the first optical transmitter 28A1-1 to the first optical receiver 30-1. The first signal path 38-1 from the first location to the second location is substantially parallel to the arm axis of the user. It is to be understood that additional optical signals transmitted by other optical transmitters 28 and received by optical transmitters 110 may travel a substantially similar signal path as the first optical signal. Signal path 38-1 is depicted in FIG. 4B using dashed lines because the signal path 38-1 are within the upper layers of skin tissue of the user under the bottom wall 32 of the wrist-worn electronic device 10 when the wrist-worn electronic device 10 is worn by the user.

In some embodiments, the first optical transmitter 28A1-1 is configured by processor 26 to transmit a first optical signal having a first wavelength, a second optical transmitter 28A1-2 configured by processor 26 to transmit a second optical signal having a second wavelength and a third optical transmitter 28A1-3 configured by processor 26 to transmit a third optical signal having a third wavelength, each optical signal separated in time such that the optical transmitter array 34 is controlled by the processor 26 to transmit the first optical signal during a first period of time, the second optical signal during a second period of time and the third optical signal during a third period of time. Such time divisional multiplexing (TDM) techniques enable the processor 26 to control the specific time period (one of a plurality of predetermined time windows) during which each of the plurality of optical transmitters may transmit an optical signal having a particular wavelength into the user's skin. As the number of sequential optical transmissions increase, the duration of time required for all transmissions to occur sequentially increases, which extends the minimum period of time between successive optical signal transmissions in a transmission sequence.

In other embodiments, the processor 26 may utilize techniques to enable the first optical transmitter 28A1-1, the second optical transmitter 28A1-2 and the third optical transmitter 28A1-3 of optical transmitter array 34 to transmit a first optical signal having a first wavelength, a second optical signal having a second wavelength and a third optical signal having a third wavelength simultaneously (all three optical signals being transmitted during first time period, a second time period and/or a third time period) or at partially overlapping time periods (one or more of the three optical signals being transmitted during first time period, a second time period and/or a third time period). For instance, the processor 26 may implement the modulation of each optical signal at different (non-overlapping), predetermined frequencies, signal phases and/or waveforms to enable the multiplexing of a plurality of optical signals. Such modulation techniques enable the processor 26 to control modulators to generate carrier signals having different frequencies and a plurality of optical transmitters 28 to enable all of the optical transmitters 28 to output optical signals simultaneously either continuously or for a predetermined duration of time. Details about and examples related to such modulation techniques are provided in application Ser. No. 18/637,928, entitled “Use of Frequency Division Multiplexing for Optical Cardiac Signals,” filed on Apr. 17, 2024, which is incorporated by reference in its entirety.

An optical receiver 30 measures an intensity of the received (reflected from the user's skin) first optical signal, the second optical signal and the third optical signal, and generates a first PPG signal corresponding to the measured intensity of the first optical signal, a second PPG signal corresponding to the measured intensity of the second optical signal and a third PPG signal corresponding to the measured intensity of the third optical signal. The intensity of the first optical signal, the second optical signal and the third optical signal varies in accordance with the amount of blood in the regions of the user's tissue in the signal path 38 and changes as the blood is moved through the body of the user with each heartbeat. In addition, the intensity of the first optical signal, the second optical signal and the third optical signal varies in accordance with composition of blood (relative amounts of, for example, oxygenated, reduced and glycated hemoglobins in red blood cells). The changing levels and composition of blood in the tissue of the skin of the user proximate to the optical transmitter and receiver assembly 36 along the signal path 38 results in different intensity of the first, second and third optical signals, respectively. Accordingly, the processor 26 can determine physiological information for the user based on the first PPG signal, the second PPG signal and/or the third PPG signal.

In embodiments, an optical receiver 30 receives optical signals output by a plurality of optical transmitter arrays 34. For example, the second optical signal may be output from an optical transmitter 28 of a second optical transmitter array 34-2 and reflected from the upper layers of skin of the user towards the optical receiver 30 along a second signal path 38-2. Similarly, the third optical signal may be output from an optical transmitter of a third optical transmitter array 34-3 and reflected from the upper layers of skin of the user towards the optical receiver 30 along a third signal path 38-3. The optical receiver 30 measures an intensity of the first optical signal, the second optical signal and the third optical signal, and generates a first PPG signal corresponding to the measured intensity of the first optical signal, a second PPG signal corresponding to the measured intensity of the second optical signal and a third PPG signal corresponding to the measured intensity of the third optical signal. It is to be understood that, in some embodiments, a second optical receiver 30-2 proximate to the first transmitter array 34-1, the second transmitter array 34-2 and the third transmitter array 34-3 may receive one or more of the first optical signal, the second optical signal and the third optical signal output by the first transmitter array 34-1, the second transmitter array 34-2 and the third transmitter array 34-3, respectively, with at least one optical signal traveling along a second signal path 38-2.

In embodiments, the first optical receiver 30-1 is separated from (relative to) the first individual optical transmitter 28-1 such that the first optical signal transmitted from (output by) the first individual optical transmitter 28-1 travels to the first optical receiver 30-1 along signal path 38-4 that is substantially parallel to the arm axis of the user. The arm axis extends along a portion of a length of an arm of the user from an elbow to a hand of that arm. The second optical receiver 30-2 may be separated from the second individual optical transmitter 28-2 such that the second optical signal transmitted from (output by) the second individual optical transmitter 28-2 travels to the second optical receiver 30-2 along signal path 38-8 that is substantially parallel to the arm axis of the user.

In embodiments, a first lens is positioned along bottom wall 32 at the first location over the first optical transmitter 28A1-1 (within the first optical transmitter array 34-1) and a second lens is positioned along bottom wall 32 at the second location over the first optical transmitter 28A2-1. In such embodiments, if a plurality of optical signals are output by the first optical transmitter 28A1-1, the plurality of optical signals pass through the first lens and are received by the first optical receiver 30-1. Similarly, if a plurality of optical signals are output by the second optical transmitter 28A2-1, the plurality of optical signals pass through the second lens and are received by the second optical receiver 30-2. FIG. 4B illustrates one of a plurality of signal paths 112 that may be substantially parallel to a user's arm axis.

Referring to FIGS. 2A, 2B, 3A, and 3B, the optical transmitter and receiver assembly 36 includes a first optical transmitter array 34-1, a second optical transmitter array 34-2, and an Nth optical transmitter array 34-N. Each optical transmitter array 34 includes a first optical transmitter 28A #-1, a second optical transmitter 28A #-2, and a third optical transmitter 28A #-3, wherein “#” refers to the number of the optical transmitter array 34. The optical transmitter and receiver assembly 36 includes a first individual optical transmitter 28-1, a second individual optical transmitter 28-2, and an Nth individual optical transmitter 28-N. The optical transmitter and receiver assembly 36 further includes a first optical receiver 30-1, a second optical receiver 30-2, and an Nth optical receiver 30-N. In addition, the optical transmitter and receiver assembly 36 includes first through fourth signal paths 38-1 through 38-4 for the first optical transmitter array 34-1, the first individual optical transmitter 28-1, and the first optical receiver 30-1. The optical transmitter and receiver assembly 36 includes fifth through eighth signal paths 38-5 through 38-8 for the second optical transmitter array 34-2, the second individual optical transmitter 28-2, and the second optical receiver 30-2. And, the optical transmitter and receiver assembly 36 includes first through fourth signal paths 38N-1 through 38N-4 for the Nth optical transmitter array 34-N, the Nth individual optical transmitter 28-N, and the Nth optical receiver 30-N.

In exemplary embodiments, as shown in FIGS. 2A and 3A, multiple combinations of the optical transmitter array 34, the optical transmitter 28, and the optical receiver 30 are positioned adjacent to one another (vertically) to form a column. In other embodiments, multiple combinations of the optical transmitter array 34, the optical transmitter 28, and the optical receiver 30 may be positioned adjacent to one another (horizontally) to form a row. Or, multiple combinations of the optical transmitter array 34, the optical transmitter 28, and the optical receiver 30 may be positioned adjacent to one another in two dimensions (vertically and horizontally) to form a grid.

Other embodiments of the electronic fitness device 10 include a combination of the optical transmitter array 34, the optical transmitter 28, and the optical receiver 30 as shown in FIGS. 2B and 3B. A first portion of the optical transmitter-receiver combination may be positioned with a first orientation, such that the optical receiver 30 is positioned to the right (as shown) of the optical transmitter array 34 and the optical transmitter 28. A second portion of the optical transmitter-receiver combination may be positioned with a second orientation, such that the optical receiver 30 is positioned to the left (as shown) of the optical transmitter array 34 and the optical transmitter 28.

Pairing each optical transmitter array 34 with an optical transmitter 28 enables the processor 26 to control the optical transmitters 28 (within the optical transmitter array 34) to output optical signals having a first wavelength, optical signals having a second wavelength, and optical signals having a third wavelength from a common location and to control a corresponding individual optical transmitter 28 to output an optical signal having a fourth wavelength from a center location closer to the optical receiver 30. The processor 26 controlling the optical elements to transmit and receive optical signals having four wavelengths enables the transmission and receipt of optical signals in any of the four wavelength bands that are shown in FIG. 7.

The electronic fitness device 10 may operate as follows. The user wears the electronic fitness device 10, which is secured by the wrist band 14 on the user's wrist. Securing the electronic fitness device 10 on the user's wrist causes the housing 12 to apply pressure on the user's skin and securing the housing 12 too tightly may cause the housing 12 to apply substantial pressure on the user's skin that may compress the blood vessels of the user in the area of the tissue through which optical signals pass from the optical transmitters 28 to one or more optical receivers 30. The user may manually request a measurement of a physiological metric, such as a pulse oximetry level or a hemoglobin check, or the processor 26 may be programmed or configured to automatically or periodically make a measurement of one or more physiological metrics, such as pulse oximetry or hemoglobin components.

The processor 26 controls one or more individual optical transmitters 28 and/or one or more optical transmitter arrays 34, such as those shown in FIGS. 2A, 2B, 3A, and 3B, to output optical signals. To determine a physiological metric that typically requires data from PPG signals associated with optical signals having two wavelengths, such as pulse oximetry, the processor 26 controls two optical transmitters 28 to output two optical signals for use with determining the physiological metric such as pulse oximetry and the processor 26 controls a third optical transmitter 28 to output a third optical signal for use with determining a pressure metric value (PMV) with one of the two optical signals that are used to determine the physiological metric. In addition, the processor 26 may select optical transmitters 28 which can emit optical signals having wavelengths in Bands 2, 3, and 4, as shown in FIG. 7. The processor 26 may control the optical transmitters 28 using transmitter electronic signals. For example, each transmitter electronic signal may have a first level that results in a respective one of the optical transmitters 28 turning on to emit its respective optical signal. Similarly, each transmitter electronic signal may have a second level that results in a respective one of the optical transmitters 28 turning off. Accordingly, the processor 26 may control a first optical transmitter 28 to emit a first optical signal having a first wavelength for a first period of time, a second optical transmitter 28 to emit a second optical signal having a second wavelength for a second period of time, and a third optical transmitter 28 to emit a third optical signal having a third wavelength for a third period of time, wherein the first, second, and third periods of time may be equivalent and non-overlapping.

The optical signals output by the optical transmitters 28 travel through the user's skin and tissue where the signals pass through and/or reflect from the user's blood vessels such that the optical signals are modified, influenced, impacted or otherwise modulated by from, and transmitting through, the blood vessels and take on PPG signal waveform characteristics. The modified, influenced, impacted or otherwise modulated optical signals exit the skin and are received by at least one optical receiver 30, which outputs a plurality of PPG signals, such as the one shown in FIG. 5. For example, the optical receiver 30 outputs, or generates, a first PPG signal resulting from receiving the first optical signal, a second PPG signal resulting from receiving the second optical signal, and a third PPG signal resulting from receiving the third optical signal. In various embodiments with reference to FIGS. 2A, 2B, 3A, and 3B, multiple optical receivers 30 may receive the modulated optical signals from groups of optical transmitters 28 and/or optical transmitter arrays 34, wherein each optical receiver 30 outputs the first, second, and third PPG signals.

The processor 26 receives the PPG signals from the optical receiver 30. The processor 26 follows the PPG signal analysis process detailed above and summarized here. The processor 26 is configured to determine the AC value and the DC value of each PPG signal. Using the AC and DC values from two PPG signals resulting from optical signals having different wavelengths, such as 840 nm and 940 nm, the processor 26 is configured to determine the pressure metric value (PMV) based on the determined AC and DC values corresponding to each wavelength of EQ. 8. Using the values of the two wavelengths and the PMV, the processor 26 is configured to determine the pressure compensation factor (PCF). In embodiments, the processor 26 may be configured to retrieve the PCF from a lookup table by substantially matching the two wavelengths used to determine the PMV and the determined PMV to PCF values in the lookup table. Or, in other embodiments, the processor 26 may determine the PCF based on the two wavelengths used to determine the PMV and the determined PMV into one or more mathematical equations to determine the PCF. Alternatively, the processor 26 may execute an algorithm which utilizes the two wavelengths used to determine the PMV and the determined PMV to determine the PCF. The processor 26 also determines the physiological metric of interest (without pressure compensation) using the data from applicable PPG signals. For example, if the user has provided a user input to the user interface 18 to determine a pulse oximetry, then the processor 26 is configured to determine and uses the data from the PPG signals associated with optical signals having a wavelength of 660 nm and optical signals having a wavelength of 940 nm with EQ. 11 to determine the (uncompensated) pulse oximetry. Once the physiological metric and the PCF are determined, the processor 26 is configured to determine the pressure-compensated physiological metric as a mathematical function of the two values, such as a function of a product of the uncompensated pulse oximetry and the PCF or a function of a sum of (addition to or subtraction from) the PCF and the uncompensated pulse oximetry.

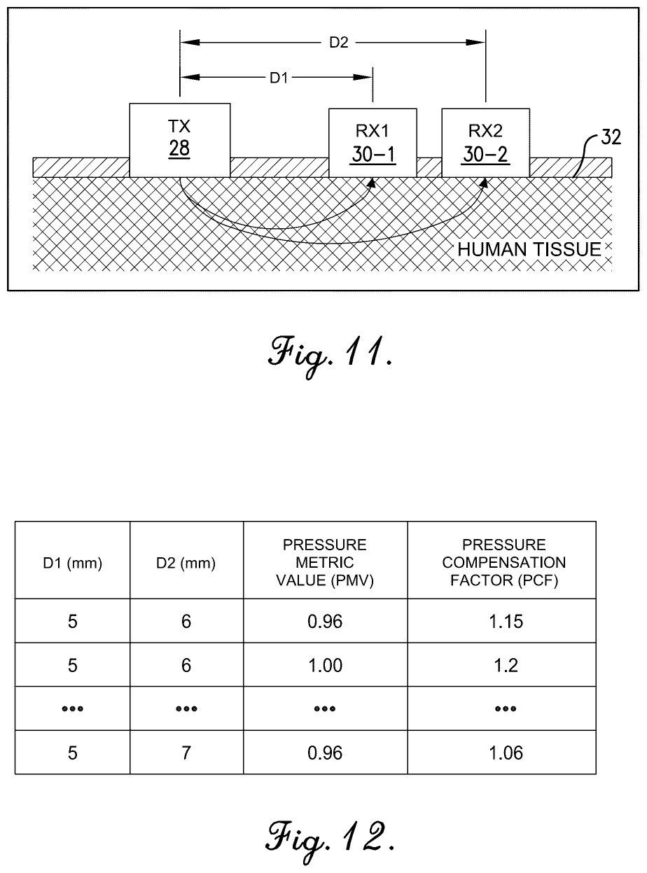

Referring to FIG. 11, other embodiments of the current technology relate to a determination of PPG waveform characteristics, such as the AC value and the DC value, for PPG signals resulting from optical signals that travel over different path lengths (D1 and D2) within the user's skin and tissue. In such embodiments, the processor 26 may be configured to account (compensate) for a substantial application of force to the skin tissue that results in compression of the blood vessels that may adversely impact the accuracy of physiological metrics and information determined by the processor for the user based on the PPG signals by determining a path length pulse ratio corresponding to extravascular tissue pressure based on the pressure metric value for two optical signals of the same (common) wavelength (λ1) and using a determined path length pulse ratio to determine a pressure metric value and adjust blood-related and cardiac physiological metrics and information determined for a user. Specifically, the processor 26 may be configured to determine a path length pulse ratio-based pressure metric value using the pressure metric value (common wavelength) (EQ. 9), wherein λ1 is common to all portions of the quotient shown in EQ. 13:

$$Pressure Metric Value ( Common Wavelength ) = \frac{{AC}_{λ 1} ( D 2 ) / {DC}_{λ 1} ( D 2 )}{{AC}_{λ 2} ( D 1 ) / {DC}_{λ 2} ( D 1 )} \qquad EQ . 13$$

As detailed above, the processor 26 may determine a pressure metric value and a pressure compensation factor based on a relationship stored in the memory element 24, and a pressure-compensated physiological metrics and information, such as glycated hemoglobin (HbA1c) level or pulse oximetry level (blood oxygen saturation).

In embodiments, the processor may be configured to determine a pressure metric value and a pressure compensation factor based on the optical layout and a first distance between an optical transmitter 28 and a first optical receiver 30-1 and a second distance between the optical transmitter 28 and a second optical receiver 30-2. As shown in FIG. 11, a representative optical sensor geometry associated with PPG signals produced by optical receivers 30-1, 30-2 are used to generate a pressure metric value (PMV) based on the pressure metric value (Common Wavelength) for two optical signals having the same (common) wavelength that travel from an optical transmitter 28 along signal paths to at least two optical receivers 30-1, 30-2 positioned at different distances from the optical transmitter. The spacing of (separation distances between) optical transmitter 28, which outputs the optical signals, to the first optical receiver 30-1 is different from the spacing of (separation distances between) that optical transmitter 28 to the second optical receiver 30-2.

In such embodiments related to the path length pulse ratio pressure metric, the pressure metric is determined based on the pressure metric value (Common Wavelength) above, where the measurements based on signals output by first optical receiver 30-1 correspond to the distance between the optical transmitter and first optical receiver 30-1 (labeled “D1”) and the measurements based on signals output by second optical receiver 30-2 correspond to the distance between the optical transmitter and second optical receiver 30-2 (labeled “D2”). However, it is to be understood that the pressure metric value (Common Wavelength) could utilize the inverted relationship, where a first quotient of the AC value and the DC value at a shorter distance (D1) is divided by a second quotient of the AC value and the DC value at a longer distance (D2). Additionally, in some embodiments, the pressure metric value (Common Wavelength) could instead utilize two optical transmitters and one optical receiver that form two signal paths of different distances.

In some embodiments, a wavelength λ1 of 700 nm is associated with the optical signals output by the optical receiver 28 and associated with PPG signals output by the optical receivers 30-1, 30-2 that are used by the processor 26 to determine the path length pulse ratio pressure metric. In other embodiments, any wavelength λ1 between 600 nm and 1500 nm is associated with the optical signals output by the optical receiver 28 and associated with PPG signals output by the optical receivers 30-1, 30-2 that are used by the processor 26 to determine the path length pulse ratio pressure metric.

In an embodiment, the processor 26 is configured to determine a pressure metric value (Common Wavelength) based on multiple sets of PPG signal data, each set generated by optical receivers positioned at different distances from an optical transmitter (where distance pairs D1, D2 are different for each set) or using optical signals having different wavelengths (the optical signals positioned substantially the same distances (D1 and D2) from the optical transmitter for each set, but each set utilizing different wavelength). In embodiments, a pressure metric value (Common Wavelength) is calculated at each wavelength used in pulse spectrometry analysis. In an exemplary embodiment, for pressure metric value (Common Wavelength) using λ1 of 660 nm and 940 nm, a first pressure metric value (Common Wavelength) determination is made for a λ1 of 660 nm and a second pressure metric value (Common Wavelength) determination is made for a λ1 of 940 nm.

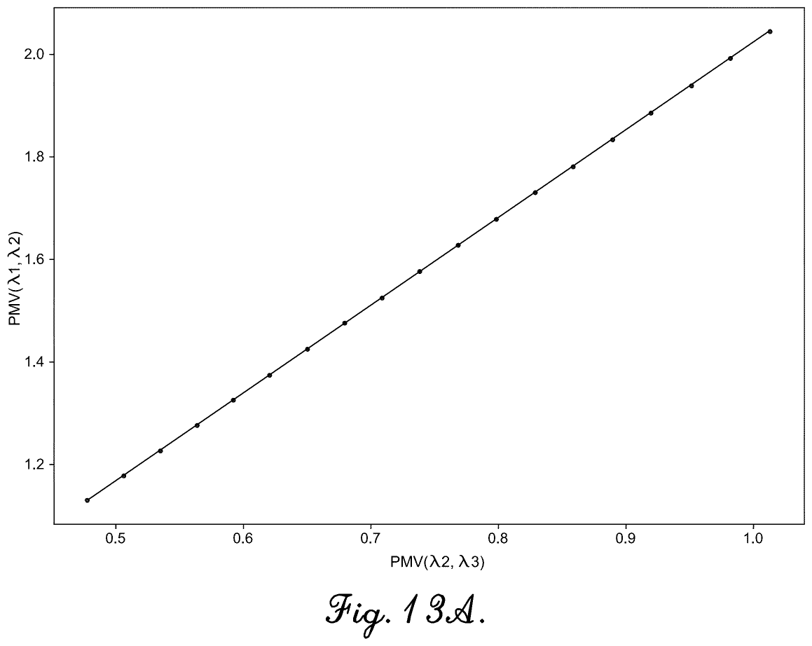

In embodiments, the memory element 24 of the electronic fitness device 10 may store a relationship that associates a pressure compensation factor (PCF) with a plurality of pressure metric values (PMV) associated with external skin tissue pressure and a plurality of distances, or path lengths, between the optical transmitter 28 and the optical receivers 30 for a given optical signal, such as the embodiment shown in FIG. 11. For instance, with reference to FIG. 12, the memory element 24 may store a lookup table that includes four columns and a plurality of rows of data. The four columns include the first distance D1 between one optical transmitter 28 and a first optical receiver 30, the second distance D2 between the one optical transmitter 28 and a second optical receiver 30, the PMV, and the PCF. Each row includes the PCF value which is associated with a combination of respective values of the first distance D1, the second distance D2, and the PMV. The first distance D1, the second distance D2, and the PMV are used as inputs, keys, or independent variables to match the respective values in a given row of the table. The PCF, associated with the respective values of the first distance D1, the second distance D2, and the PMV, is read out from the memory element 24. The processor 26 may determine a pressure metric value associated with the pressure applied to a user's extremity and, based on the relationship stored in the memory element 24, determine a pressure compensation factor for two or more distance sets of interest, wherein D1 and D2 are the distances between one optical transmitter 28 and a first optical receiver 30-1 and the one optical transmitter 28 and a second optical receiver 30-2, respectively, that may be utilized to determine physiological metrics and information, such as a pulse oximetry level (blood oxygen saturation) or glycated hemoglobin (HbA1c) level, in order to determine pressure-compensated physiological metrics and information that accounts (compensates) for the application of force to the skin tissue by the electronic device housing 12.

Referring to FIGS. 11 and 12, the electronic fitness device 10 may operate as follows. The user wears the electronic fitness device 10, which is secured by the wrist band 14 on the user's wrist. Securing the electronic fitness device 10 on the user's wrist may cause the housing 12 to apply pressure on the user's skin. The user may manually request a measurement of a physiological metric, such as a pulse oximetry level or a hemoglobin check, or the processor 26 may be programmed or configured to automatically or periodically make a measurement of one or more physiological metrics, such as pulse oximetry or hemoglobin components.

The processor 26 outputs at least a first transmitter electronic signal that is received by the optical transmitter 28 which emits a first optical signal to travel through the user's skin and get modified by the user's blood vessels along two different paths with two different distances, D1 and D2. The first optical receiver 30-1 receives the modified optical signal along the first path at distance D1, and the second optical receiver 30-2 receives the modified optical signal along the second path at distance D2. The first optical receiver 30-1 outputs a first PPG signal, and the second optical receiver 30-2 outputs a second PPG signal.

The processor 26 receives the first PPG signal and the second PPG signal and determines the AC value and the DC value of each signal, that is AC(D1), DC(D1), AC(D2), and DC(D2). The processor 26 enters the values into EQ. 13 and determines the PMV. With the distance D1, the distance D2, and the PMV, the processor 26 accesses the lookup table, as shown in FIG. 12, and retrieves the value of the PCF associated with the values of the distance D1, the distance D2, and the PMV.

The processor 26 also outputs one or more transmitter electronic signals that are received by optical transmitters 28 or optical transmitter arrays 34, such as those shown in FIGS. 2A, 2B, 3A, and 3B, which are configured to output optical signals having wavelengths in the appropriate Bands (Band 1, Band 2, Band 3, and/or Band 4) to determine the requested physiological metric. For example, to determine the user's pulse oximetry, the processor 26 outputs transmitter electronic signals to optical transmitters 28 or optical transmitter arrays 34 configured to output optical signals having wavelengths in Band 2 and Band 4. The processor 26 receives the corresponding PPG signals from respective optical receivers 30. The processor 26 is configured to determine the (uncompensated) physiological metric using the PPG signal data that is plugged into the appropriate equation(s) for determining the physiological metric. With the physiological metric and the PCF being determined, the processor 26 is configured to determine the pressure-compensated physiological metric as a mathematical function of the two values, such as a function of a product of the pulse oximetry and the PCF or a function of a sum of the pulse oximetry and the PCF.

As an example of the processor 26 being configured to using the relationships between sets of PMVs (e.g., a relationship between a first set of PMVs and a second set of PMVs) over a period of time over which a current pulse oximetry level (SpO2) changes for a user to determine the PCF, the processor 26 may receive over a period of time a first PPG signal associated with optical signals having a first wavelength λ1, a second PPG signal associated with optical signals having a second wavelength λ2 and a third PPG signal associated with optical signals having a third wavelength λ3, determine a plurality of sets of PMVs based on the three PPG signals, determine a relationship between the first PMV set associated with a first pair of optical signals (associated with the first wavelength λ1 and the second wavelength λ2) and a second PMV set associated with a second pair of optical signals (associated with the third wavelength λ3 and the second wavelength λ2) and determine a PCF based on the determined relationship between the PMV sets and an expected relationship between those PMV sets that is stored in memory element 24. For example, the processor 26 may determine, over a range of pulse oximetry levels (SpO2), a first set of PMVs associated with PPG signals having a first wavelength λ1 between 600-650 nm and PPG signals having a second wavelength λ2 between 820-980 nm and a second set of PMVs associated with PPG signals having a third wavelength λ3 between 650-700 and PPG signals having the second wavelength λ2 between 820-980 nm. The processor 26 may then determine, for that range of pulse oximetry levels (SpO2), a relationship between the first set of PMVs (that are associated with optical signals having the first wavelength λ1 between 600-650 nm and optical signals having the second wavelength λ2 between 820-980 nm) and the second set of PMVs (that are associated with optical signals having the third wavelength λ3 between 650-700 nm and optical signals having the second wavelength λ2 between 820-980 nm). As shown in FIG. 13B, the measured relationship between the first set of PMVs and the second set of PMVs may be visualized over a range of pulse oximetry levels (SpO2).

In some embodiments, the processor 26 determines the expected relationship between the two sets of PMVs for a range of pulse oximetry levels (SpO2) based on expected PMVs stored in memory element 24 for each wavelength, which may be based in part on physiological information for the user, such as the user's age, weight and other characteristics. In other embodiments, the memory element 24 stores the expected PMVs for a plurality of wavelength pairs and an expected relationship between at least two PMV sets of wavelength pairs and the processor 26 retrieves the expected relationship for the at least two PMV sets of wavelength pairs that are stored in the memory element 24 for comparison with the measured relationship for the at least two PMV sets of wavelength pairs.

The processor 26 may utilize the measured relationship between the first set of PMVs for the first wavelength λ1 and second wavelength λ2 pair and the second set of PMVs for the third wavelength λ3 and second wavelength λ2 pair and an expected relationship between those two sets of PMVs (for the corresponding range of pulse oximetry levels (SpO2) and optical signals having one of the first wavelength λ1, optical signals having the second wavelength λ2 and optical signals having the third wavelength λ3 to determine the PCF. For example, the processor 26 may compare the measured relationship between PMVs for the first wavelength λ1 between 600-650 nm, second wavelength λ2 between 820-980 nm and for the third wavelength λ3 between 650-700 nm, second wavelength λ2 between 820-980 nm with the corresponding expected relationship for these wavelength pairs (the first wavelength λ1 with second wavelength λ2, and the third wavelength λ1 with second wavelength λ2) to make a determination of how much pressure has been applied to the user's blood vessels in the area where the housing 12 is secured to the user and a PCF that may account (compensate) for that pressure compressing those blood vessels for use with determining cardiac monitoring for the user based on optical signals having the first wavelength λ1 and second wavelength λ2 pair and/or for use with determining cardiac monitoring for the user based on optical signals having the second wavelength λ2 and third wavelength λ3 pair.

In embodiments, the processor 26 may determine the PCF to be a ratio at a selected pulse oximetry level (SpO2) (within the range of pulse oximetry levels (SpO2) over which the relationship was determined) of the expected relationship of PMVs to the measured relationship of corresponding PMVs. For example, the processor 26 may determine the first PCF associated with the first wavelength λ1 and second wavelength λ2 pair based on a ratio of the expected relationship of corresponding PMVs between pulse oximetry levels (SpO2) of 90% and 95% and the measured relationship of corresponding PMVs between the pulse oximetry levels (SpO2) of 90% and 95%. Similarly, the processor 26 may determine the second PCF associated with the third wavelength λ3 and second wavelength λ2 pair based on a ratio of the expected relationship of corresponding PMVs between pulse oximetry levels (SpO2) of 90% and 95% and the measured relationship of corresponding PMVs between the pulse oximetry levels (SpO2) of 90% and 95%.

In embodiments, the processor may determine a first slope between the points associated with the measured relationship between the first set of PMVs for the first wavelength λ1 and second wavelength λ2 pair and the second set of PMVs for the third wavelength λ3 and second wavelength λ2 pair. Similarly, the processor may determine a second slope between the points associated with the expected relationship between the first set of PMVs for the first wavelength λ1 and second wavelength λ2 pair and the second set of PMVs for the third wavelength λ3 and second wavelength λ2 pair. In such embodiments, the processor may be configured to determine a ratio of the first slope to the second slope and multiply one or both of the first PCF and the second PCF by the determined ratio to determine a slope-adjusted first PCF and/or slope-adjusted PCF.

Once the first PCF has been determined, the processor 26 is configured to determine a pressure-compensated physiological metric determined based on the first wavelength λ1 and second wavelength λ2 pair as a function of the determined physiological metric and the determined first PCF. Similarly, once the second PCF has been determined, the processor 26 is configured to determine a pressure-compensated physiological metric determined based on the third wavelength λ3 and second wavelength λ2 pair as a function of the determined physiological metric and the determined second PCF.

In this manner, as the processor 26 receives PPG signals over a period of time over which the user's pulse oximetry level (SpO2) levels continue to change, the processor 26 is configured to determine and use an expected relationship between a first set of PMVs associated with a first wavelength pair (the first wavelength λ1 and second wavelength λ2 pair) and a second set of PMVs associated with a second wavelength pair (the third wavelength λ3 and second wavelength λ2 pair) over a range of pulse oximetry level (SpO2) levels and a measured relationship between the first set of PMVs (associated with the first wavelength pair) and the second set of PMVs (associated with the second wavelength pair) over the corresponding range of pulse oximetry level (SpO2) levels to determine a discrepancy between the expected relationship and the measured relationship at a pulse oximetry level in the range to determine a PCF for each wavelength pair (e.g., a first PCF for determining a physiological metric for the user based on the first wavelength λ1 and second wavelength λ2 pair, a second PCF for determining a physiological metric for the user based on the third wavelength λ3 and second wavelength λ2 pair, etc.). In some embodiments, one physiological metric is determined for the user based on both a first PCF for determining a physiological metric for the user based on the first wavelength λ1 and second wavelength λ2 pair and a second PCF for determining a physiological metric for the user based on the third wavelength λ3 and second wavelength λ2 pair

In embodiments, processor 26 may receive a plurality of PPG signals that correspond to optical signals that passed along two different signal paths from an optical transmitter array 34 to optical receivers 30-1, 30-2. In such embodiments, for each signal path, the processor may determine a first set of PMVs associated with PPG signals associated with optical signals having the first wavelength λ1 and PPG signals associated with optical signals having the second wavelength λ2 and a second set of PMVs associated with PPG signals associated with optical signals having the third wavelength λ3 and PPG signals associated with optical signals having the second wavelength λ2, determine a measured relationship between the first set of PMVs associated with a first wavelength pair (the first wavelength λ1 and second wavelength λ2 pair) and the second set of PMVs associated with a second wavelength pair (the third wavelength λ3 and second wavelength λ2 pair and then determine a PCF for each wavelength pair (e.g., a first PCF for determining a physiological metric for the user based on the first wavelength λ1 and second wavelength λ2 pair, a second PCF for determining a physiological metric for the user based on the third wavelength λ3 and second wavelength λ2 pair, etc.) based on the stored relationship and the measured relationship between the first set of PMVs and the second set of PMVs. As shown in FIG. 13C, the relationship of the measured first set of PMVs and the second set of PMVs may be different for the PPG signals associated with each signal path between an optical transmitter array 34 and optical receivers 30-1, 30-2. For instance, lines fit to points representing each relationship may be offset vertically and have a different slope. For each signal path and each wavelength pair, the processor 26 is configured to determine the PCF as a ratio of the expected relationship of PMVs to the measured relationship of corresponding PMVs for at least two pulse oximetry levels within the range of pulse oximetry levels (SpO2) over which the relationship was determined (e.g., 90% and 95% SpO2), as detailed above. Once the processor 26 has determined a PCF for each pair of wavelengths and each signal path, the processor 26 is configured to determine a pressure-compensated physiological metric determined based on the corresponding wavelength pair as a function of the determined physiological metric and the determined PCF. For example, the processor 26 may determine and use a first PCF for determining a physiological metric for the user based on the first wavelength λ1 and second wavelength λ2 pair along a first signal path between an optical transmitter array 34 and optical receiver 30-1, a second PCF for determining a physiological metric for the user based on the third wavelength λ3 and second wavelength λ2 pair along a first signal path between an optical transmitter array 34 and optical receiver 30-2, a third PCF for determining a physiological metric for the user based on the third wavelength λ3 and second wavelength λ2 pair along a second signal path between an optical transmitter array 34 and optical receiver 30-1 and a fourth PCF for determining a physiological metric for the user based on the third wavelength λ3 and second wavelength λ2 pair along a second signal path between an optical transmitter array 34 and optical receiver 30-2.

Throughout this specification, relational and/or directional terms, such as “above”, “below”, “up”, “upper”, “upward”, “down”, “lower”, “downward”, “top”, “bottom”, “outer”, “inner”, etc., along with orientation terms, such as “horizontal” and “vertical”, may be used. These terms retain their commonly accepted definitions and are used with reference to embodiments of the technology and the positions, directions, and orientations thereof shown in the accompanying figures. However, embodiments of the technology in practice may be positioned and oriented in other ways or move in other directions. Therefore, the terms do not limit the scope of the current technology.

Throughout this specification, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

Although the present application sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as computer hardware that operates to perform certain operations as described herein.

In various embodiments, computer hardware, such as a processor, may be implemented as special purpose or as general purpose. For example, the processor may comprise dedicated circuitry or logic that is permanently configured, such as an application-specific integrated circuit (ASIC), or indefinitely configured, such as an FPGA, to perform certain operations. The processor may also comprise programmable logic or circuitry (e.g., as encompassed within a general-purpose processor or other programmable processor) that is temporarily configured by software to perform certain operations. It will be appreciated that the decision to implement the processor as special purpose, in dedicated and permanently configured circuitry, or as general purpose (e.g., configured by software) may be driven by cost and time considerations.

Accordingly, the term “processor” or equivalents should be understood to encompass a tangible entity, be that an entity that is physically constructed, permanently configured (e.g., hardwired), or temporarily configured (e.g., programmed) to operate in a certain manner or to perform certain operations described herein. Considering embodiments in which the processor is temporarily configured (e.g., programmed), each of the processors need not be configured or instantiated at any one instance in time. For example, where the processor comprises a general-purpose processor configured using software, the general-purpose processor may be configured as respective different processors at different times. Software may accordingly configure the processor to constitute a particular hardware configuration at one instance of time and to constitute a different hardware configuration at a different instance of time.

Computer hardware components, such as communication elements, memory elements, processors, and the like, may provide information to, and receive information from, other computer hardware components. Accordingly, the described computer hardware components may be regarded as being communicatively coupled. Where multiple of such computer hardware components exist contemporaneously, communications may be achieved through signal transmission (e.g., over appropriate circuits and buses) that connect the computer hardware components. In embodiments in which multiple computer hardware components are configured or instantiated at different times, communications between such computer hardware components may be achieved, for example, through the storage and retrieval of information in memory structures to which the multiple computer hardware components have access. For example, one computer hardware component may perform an operation and store the output of that operation in a memory device to which it is communicatively coupled. A further computer hardware component may then, at a later time, access the memory device to retrieve and process the stored output. Computer hardware components may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processors may be located in a single location (e.g., within a home environment, an office environment or as a server farm), while in other embodiments the processors may be distributed across a number of locations.

Unless specifically stated otherwise, discussions herein using words such as “processing,” “computing,” “calculating,” “determining,” “presenting,” “displaying,” or the like may refer to actions or processes of a machine (e.g., a computer with a processor and other computer hardware components) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112 (f) unless traditional means-plus-function language is expressly recited, such as “means for” or “step for” language being explicitly recited in the claim(s).

Although the technology has been described with reference to the embodiments illustrated in the attached drawing figures, it is noted that equivalents may be employed and substitutions made herein without departing from the scope of the technology as recited in the claims.

Having thus described various embodiments of the technology, what is claimed as new and desired to be protected by Letters Patent includes the following:

1. An electronic fitness device comprising:
a housing including a bottom wall to be positioned at least partly in contact with a user's skin, the housing asserting a pressure on the user's skin;
a first optical transmitter positioned along the bottom wall and configured to emit a first optical signal having a first wavelength, the first optical signal directed into the skin of the user;
a second optical transmitter positioned along the bottom wall and configured to emit a second optical signal having a second wavelength, the second optical signal directed into the skin of the user;
an optical receiver positioned along the bottom wall, the optical receiver configured to:
receive the first optical signal and the second optical signal, each optical signal modulated by the skin of the user, and
generate a first photoplethysmogram (PPG) signal resulting from the received first optical signal and a second PPG signal resulting from the received second optical signal;
a memory element; and
a processor electrically coupled with the optical receiver and the memory element, the processor configured to:
receive the first PPG signal and the second PPG signal,
determine a pressure metric value based on a characteristic of a waveform of the first PPG signal and a characteristic of a waveform of the second PPG signal, and
determine a pressure compensation factor based on the determined pressure metric value, the first wavelength corresponding to the first optical signal and the second wavelength corresponding to the second optical signal.

2. The electronic fitness device of claim 1, further comprising a third optical transmitter positioned along the bottom wall and configured to emit a third optical signal having a third wavelength, the third optical signal directed into the skin of the user;
wherein the optical receiver positioned is further configured to receive the third optical signal, the third optical signal modulated by the skin of the user; and
wherein the processor is further configured to determine:
a physiological metric for the user, the physiological metric based on characteristics of the waveform of the third PPG signal and one of the first PPG signal and the second PPG signal, and
a pressure-compensated physiological metric as a function of the determined physiological metric and the determined pressure compensation factor.

3. The electronic fitness device of claim 1,
wherein the characteristic of the waveform of the first PPG signal is an AC component of the waveform of the first PPG signal and a DC component of the waveform of the first PPG signal, and
wherein the characteristic of the waveform of the second PPG signal is an AC component of the waveform of the second PPG signal and a DC component of the waveform of the second PPG signal.

4. The electronic fitness device of claim 3, wherein determining the pressure metric value includes:
determining a first AC component to DC component ratio of the waveform of the first PPG signal,
determining a second AC component to DC component ratio of the waveform of the second PPG signal, and
determining a ratio of the first AC component to DC component ratio to the second AC component to DC component ratio.

5. The electronic fitness device of claim 1, wherein the processor is further configured to determine a pressure-compensated physiological metric based on the determined pressure compensation factor.

6. The electronic fitness device of claim 1, wherein the determined pressure metric value, a value of the first wavelength corresponding to the first optical signal, a value of the second wavelength corresponding to the second optical signal, and the determined pressure metric value are stored in the memory element in association with the identified pressure compensation factor.

7. The electronic fitness device of claim 1, wherein the first wavelength ranges from approximately 900 nanometers to approximately 980 nanometers.

8. The electronic fitness device of claim 1, wherein the second wavelength ranges from approximately 820 nanometers to approximately 900 nanometers.

9. An electronic fitness device comprising:
a housing including a bottom wall to be positioned at least partly in contact with a user's skin;
a first optical transmitter positioned along the bottom wall and configured to emit a first optical signal having a first wavelength, the first optical signal directed into the skin of the user;
a second optical transmitter positioned along the bottom wall and configured to emit a second optical signal having a second wavelength, the second optical signal directed into the skin of the user;
a third optical transmitter positioned along the bottom wall and configured to emit a third optical signal having a third wavelength, the third optical signal to be directed into the skin of the user;
an optical receiver positioned along the bottom wall, the optical receiver configured to:
receive the first optical signal, the second optical, and the third optical signal, each optical signal modulated by the skin of the user, and
generate a first photoplethysmogram (PPG) signal resulting from the received first optical signal, a second PPG signal resulting from the received second optical signal, and a third PPG signal resulting from the received third optical signal, each received optical signal including a cardiac component of the user;
a memory element configured to store a plurality of pressure compensation factors, each pressure compensation factor associated with a pressure metric value, the first optical signal having the first wavelength and the second optical signal having the second wavelength; and
a processor electrically coupled with the memory element and configured to:
receive the first PPG signal, the second PPG signal, and the third PPG signal,
determine a pressure metric value based on a characteristic of a waveform of the first PPG signal and a characteristic of a waveform of the second PPG signal,
identify, and retrieve from the memory element, one of the plurality of pressure compensation factors based on the determined pressure metric value, and
determine a pressure-compensated physiological metric based on characteristics of the waveform of the third PPG signal and the identified pressure compensation factor.

10. The electronic fitness device of claim 9, wherein the first wavelength ranges from approximately 900 nanometers to approximately 980 nanometers.

11. The electronic fitness device of claim 9, wherein the second wavelength ranges from approximately 820 nanometers to approximately 900 nanometers.

12. The electronic fitness device of claim 9, wherein the third wavelength ranges from approximately 500 nanometers to approximately 800 nanometers.

13. The electronic fitness device of claim 9, wherein
the characteristic of the waveform of the first PPG signal is an AC component of the waveform of the first PPG signal and a DC component of the waveform of the first PPG signal,
the characteristic of the waveform of the second PPG signal is an AC component of the waveform of the second PPG signal and a DC component of the waveform of the second PPG signal, and
the characteristic of the waveform of the third PPG signal is an AC component of the waveform of the third PPG signal and a DC component of the waveform of the third PPG signal.

14. The electronic fitness device of claim 9, wherein determining the pressure metric value includes
determining a first AC component to DC component ratio of the waveform of the first PPG signal,
determining a second AC component to DC component ratio of the waveform of the second PPG signal, and
determining a ratio of the first AC component to DC component ratio to the second AC component to DC component ratio.

15. The electronic fitness device of claim 9, wherein the determined pressure-compensated physiological metric is a pressure-compensated pulse oximetry for the user, the pressure-compensated pulse oximetry being a function of a product of the identified pressure compensation factor and a ratio of:
the first AC component to DC component ratio of the waveform of the first PPG signal, and
the third AC component to DC component ratio of the waveform of the third PPG signal.

16. The electronic fitness device of claim 9, wherein the determined pressure-compensated physiological metric is a pressure-compensated pulse oximetry for the user, the pressure-compensated pulse oximetry being a function of a sum of the identified pressure compensation factor and a ratio of:
the first AC component to DC component ratio of the waveform of the first PPG signal, and
the third AC component to DC component ratio of the waveform of the third PPG signal.

17. An electronic fitness device comprising:
a housing including a bottom wall to be positioned at least partly in contact with a user's skin;
a first optical transmitter positioned along the bottom wall and configured to emit a first optical signal having a first wavelength, the first optical signal directed into the skin of the user;
a second optical transmitter positioned along the bottom wall and configured to emit a second optical signal having a second wavelength, the second optical signal directed into the skin of the user;
a third optical transmitter positioned along the bottom wall and configured to emit a third optical signal having a third wavelength, the third optical signal directed into the skin of the user;
an optical receiver positioned along the bottom wall, the optical receiver configured to:
receive the first optical signal, the second optical, and the third optical signal, each optical signal modulated by the skin of the user, and
generate a first photoplethysmogram (PPG) signal resulting from the received first optical signal, a second PPG signal resulting from the received second optical signal, and a third PPG signal resulting from the received third optical signal, each received optical signal including a cardiac component of the user;
a memory element configured to store a plurality of pressure compensation coefficients, each pressure compensation coefficient associated with a pressure metric value and the first optical signal having the first wavelength and the second optical signal having the second wavelength; and
a processor electrically coupled with the optical receiver and the memory element, the processor configured to:
receive the first PPG signal, the second PPG signal, and the third PPG signal,
determine a pressure metric value based on a characteristic of a waveform of the first PPG signal and a characteristic of a waveform of the second PPG signal,
retrieve from the memory element pressure compensation coefficients associated with the first wavelength associated with the first optical signal and the second wavelength associated with the second optical signal,
determine a pressure compensation factor based on the pressure compensation coefficients, the first PPG signal, and the second PPG signal, and
determine a pressure-compensated physiological metric based on characteristics of the waveform of the third PPG signal and the determined pressure compensation factor.

18. The electronic fitness device of claim 17, wherein
the characteristic of the waveform of the first PPG signal is an AC component of the waveform of the first PPG signal and a DC component of the waveform of the first PPG signal,
the characteristic of the waveform of the second PPG signal is an AC component of the waveform of the second PPG signal and a DC component of the waveform of the second PPG signal, and
the characteristic of the waveform of the third PPG signal is an AC component of the waveform of the third PPG signal and a DC component of the waveform of the third PPG signal.

19. The electronic fitness device of claim 17, wherein determining the pressure metric value includes
determining a first AC component to DC component ratio of the waveform of the first PPG signal,
determining a second AC component to DC component ratio of the waveform of the second PPG signal, and
determining a ratio of the first AC component to DC component ratio to the second AC component to DC component ratio.

20. The electronic fitness device of claim 17, wherein the first wavelength ranges from approximately 900 nanometers to approximately 980 nanometers, the second wavelength ranges from approximately 820 nanometers to approximately 900 nanometers, and the third wavelength ranges from approximately 500 nanometers to approximately 800 nanometers.
